SEQUENCE LISTING (1 sequence)

SEQ ID NO: 1 - protein, 89 aa, Artificial
  Pro-piece of MBP
LHLRSETSTFETPLGAKTLPEDEETPEQEMEETPCRELEEEEEWGSGSEDASKKDGAVESISVPDMVDKNLTCPEEEDTVKVVGIPGCQ

(19) United States
(12) Patent Application Publication
(10) Pub. No.: US 2010/0184664 A1
(43) Pub. Date: Jul. 22, 2010

(54) OPHTHALMIC COMPOSITIONS USEFUL FOR IMPROVING VISUAL ACUITY

(75) Inventors: Peter A. Simmons, Yorba Linda, CA (US); Joseph Vehige, Laguna Niguel, CA (US)

Correspondence Address: ALLERGAN, INC., 2525 DUPONT DRIVE, T2-7H, IRVINE, CA, 92612-1599, US

(73) Assignee: Allergan, Inc.

(21) Appl. No.: 12/574,212

(22) Filed: Oct. 6, 2009

Related U.S. Application Data
(60) Provisional application No. 61/106,889, filed on Oct. 20, 2008.

(51) Int. Cl. A61K 38/16; A61K 31/205; A61K 31/717; A61P 27/04

(52) U.S. Cl. 514/12; 514/556; 514/57

(57) ABSTRACT

The present invention provides a method of improving the visual acuity of a person in need thereof which comprises topically administering to said person, in an effective amount, an ophthalmic composition comprising an aqueous carrier component; and an effective amount of a tonicity component comprising a material selected from a combination of compatible solute agents, wherein said combination of compatible solute agents comprises two polyol components and one amino acid component and wherein said polyol components are erythritol and glycerol and said amino acid component is carnitine.

### CROSS REFERENCE

This application claims the benefit of U.S. Provisional Patent Application Ser. No. 61/106,889, filed on Oct. 20, 2008, the entire disclosure of which is incorporated herein by this specific reference.

### BACKGROUND OF THE INVENTION

1. Field of the Invention

The present invention relates to ophthalmic compositions and methods useful for treating eyes to improve visual acuity. More particularly, the present invention relates to ophthalmic compositions including mixtures of components which are is effective in providing the desired prevention of loss in visual acuity to human or animal eyes, and to methods for treating human or animal eyes to improve and/or prevent the loss of visual acuity by using said ophthalmic compositions.

2. Background of the Art

To resolve detail, the eye's optical system has to project a focused image on the fovea, a region inside the macula having the highest density of cone photoreceptors, thus having the highest resolution and best color vision. Acuity and color vision, despite being done by the same cells, are different physiologic functions that don't interrelate except by position. Acuity and color vision can be affected independently.

Light travels from the fixation object to the fovea through an imaginary path called the visual axis. The eye's tissues and structures that are in the visual axis and the tissues adjacent to it affect the quality of the image. These structures are: tear film, cornea, anterior chamber, pupil, lens, vitreous, and finally the retina. The posterior part of the retina, called the retinal pigment epithelium (RPE) is responsible for absorbing light that crosses the retina so it cannot bounce to other parts of the retina.

Visual acuity is affected by the size of the pupil. Optical aberrations of the eye that decrease visual acuity are at a maximum when the pupil is largest (about 8 mm), which occurs in low-light conditions. When the pupil is small (1-2 mm), image sharpness may be limited by diffraction of light by the pupil. Between these extremes is the pupil diameter that is generally best for visual acuity in normal, healthy eyes, i.e. 3 or 4 mm.

If the optics of the eye were otherwise perfect, theoretically acuity would be limited by pupil diffraction which would be a diffraction-limited acuity of 0.4 minutes of arc (minarc) or 20/8 acuity. The smallest cone cells in the fovea have sizes corresponding to 0.4 minarc of the visual field, which also places a lower limit on is acuity.

In patients with optical problems, such as cataracts, the health of the retina is assessed before subjecting the eye to surgery.

Any pathological process in the visual system will often cause decreases in visual acuity. Thus, measuring visual acuity is a simple test for accessing the health of the eyes, the visual brain, or pathway to the brain. Any relatively sudden decrease in visual acuity is always a cause for concern. Common causes of decreases in visual acuity are cataracts and scarred corneas, which affect the optical path, diseases that affect the retina, such as macular degeneration and diabetes, diseases affecting the optical path to the brain such as tumors and multiple sclerosis, and diseases affecting the visual cortex such as tumors and strokes

The spatial resolution of the visual system is usually assessed using a simple measure of static visual acuity. A typical visual acuity test consists of a number of high contrast, black-on-white targets of progressively smaller size. The smallest target that one can successfully read denotes one's visual acuity. For example, if the smallest letters that you can read upon a Snellen Eye Chart subtend 5 minutes of arc (minarc) in height, you are said to have 20/20 (or “normal”) acuity. That is, the smallest letter that your visual system can successfully resolve (at twenty feet) is 5 minarc in height.

Visual acuity is a common measure of visual status because: (1) it is easy to measure and (2) small amounts of refractive error in the eye yield marked declines in acuity test performance. Fortunately, most sources of refractive error are correctable via glasses or contact lenses.

Visual spatial processing is organized as a series of parallel—but independent—channels in the nervous system; each “tuned” to targets of a different size. As a result of this parallel organization of the visual nervous system, visual acuity measurements no longer appear to adequately describe the spatial visual abilities of a given individual. Modern vision research has clearly demonstrated that the capacity to detect and identify spatial form varies widely as a function of target size, contrast, and spatial orientation As a consequence of the above, a simple assessment of visual acuity often does not predict an individual's ability to detect objects of larger size.

Contrast sensitivity testing complements and extends the assessment of visual function provided by simple acuity tests. Contrast sensitivity measurements yield information about an individual's ability to see low-contrast targets over an extended range of target size and orientation.

Contrast sensitivity tests use sine-wave gratings as targets instead of the letters typically used in a tests of acuity. Sine-wave gratings possess useful mathematical properties and researchers have discovered that early stages of visual processing are optimally “tuned” to such targets.

A contrast sensitivity assessment procedure consists of presenting the observer with a sine-wave grating target of a given spatial frequency (i.e., the number of sinusoidal luminance cycles per degree of visual angle). The contrast of the target grating is then varied while the observer's contrast detection threshold is determined. Typically, contrast thresholds of this sort are collected using vertically oriented sine-wave gratings varying in spatial frequency from 0.5 (very wide) to 32 (very narrow) cycles per degree of visual angle.

Under normal conditions, the ocular surface of a human or animal eye is bathed in tears of a normal osmotic strength, for example, substantially isotonic. If this osmotic strength is increased, cells on the ocular surface are exposed to a hyperosmotic or hypertonic environment resulting in adverse reduction in cell volume due to trans-epithelial water loss, and other undesired changes. The compensatory mechanisms are limited, in many respects, leading to ocular surface compromise and discomfort. For example, the cells may attempt to balance osmotic pressure by increasing internal electrolyte concentration. However, at elevated electrolyte levels, cell metabolism is altered in many ways, including the reduction in enzyme activity and membrane damage. In addition, a hypertonic environment has been shown to be pro-inflammatory to the ocular surface.

The cells of many life forms can compensate for hypertonic conditions through the natural accumulation or manufacture of so-called “compatible solutes” that work like electrolytes to balance osmotic pressure yet do not interfere with cellular is metabolism like electrolytes. Compatible solutes or compatible solute agents, generally, are uncharged, can be held within a living cell, for example, an ocular cell, are of relatively small molecular weight and are otherwise compatible with cell metabolism. Compatible solutes are also considered to be osmoprotectants since they may allow cell metabolism and/or enhance cell survival under hypertonic conditions that would otherwise be restricting.

For example, a class of organisms called halophiles exist that inhabit hypersaline environments such as salt lakes, deep sea basins, and artificially-created evaporation ponds. These organisms may be eukaryotic or prokaryotic, and have mechanisms for synthesizing and/or accumulating a variety of compatible solute agents, including polyols, sugars, and amino acids and their derivatives such as glycine, betaine, proline, ectoine, and the like.

Glycerin (glycerol) is a widely used osmotic agent that has been identified as a compatible solute in a variety of cells from a number of different species. It is also regarded as a humectant and ophthalmic lubricant. In the U.S., it is applied topically to the ocular surface to relieve irritation at concentrations up to 1%, and has been used at higher concentrations to impart osmotic strength in prescription medications. Given its small size and biological origin, it should easily cross cell membranes, and transport channels have been recently identified in some cell types to facilitate glycerol movement.

Although glycerol may serve as the sole compatible solute, it may be excessively mobile, that is, cross membranes too freely, to provide an extended benefit in certain systems. An example is the human tear film where natural levels of glycerol are low. When a topical preparation is applied, migration into the cell is likely to occur fairly rapidly. However, as concentration in the tear falls, glycerol may be lost over time from cell to tear film, limiting the duration of benefit.

Another major class of compounds with osmoprotective properties in a variety of tissues is certain amino acids. In particular, betaine (trimethyl glycine) has been shown to be actively taken up by renal cells in response to osmotic challenge, and taurine is accumulated by ocular cells under hypertonic conditions.

Hypotonic compositions have been used on eyes as a method to counteract the effects of hypertonic conditions. These compositions effectively flood the ocular surface with water, which rapidly enters cells when supplied as a hypotonic artificial tear. Due to the rapid mobility of water into and out of cells, however, any benefit of a hypotonic composition will be extremely short-lived. Further, it has been demonstrated that moving cells from a hypertonic environment to an isotonic or hypotonic environment down-regulates transport mechanisms for cells to accumulate compatible solutes. Thus, use of a hypotonic artificial tear reduces the ability of cells to withstand hypertonicity when it returns shortly after drop instillation.

The tear film of the presumed normal human or animal eye may have elevated (detectable) levels of Major Basic Protein (MBP) whereas it was previously believed that this protein was only expressed under conditions of allergy with eosinophilic involvement (late phase allergy). MBP is now recognized to be produced by Mast Cells (MC) as well as eosinophils, which are known to commonly reside within ocular surface tissues and are recognized to de-granulate, releasing MBP and other cationic compounds, under antigenic stimulation, mechanical trauma, and other conditions.

Another group of cationic proteins active on the ocular surface are one or more of the defensins, which are normally part of the body's antimicrobial defense system. Defensins are found at increased levels in the tear film of dry eye patients, and may either directly or through interaction with other substances have adverse effects on the health of the ocular surface.

The primary use of artificial tears is to provide temporary relief of symptoms of discomfort associated with dry eye. Dry eye is a multifactorial disease of the tears and ocular surface that results in symptoms of discomfort, visual disturbance, and tear film instability with potential damage to the ocular surface. Artificial tears cause transient blur, proportional to product viscosity. Increased viscosity of artificial tears will prolong contact time of bulk fluid on the ocular surface, but will also induce greater visual complaints in both magnitude and duration of blur associated with use of product. Ideally, artificial tears should have a sufficiently enhanced viscosity to provide longer lasting lubricating and moisturizing benefits, but this enhanced viscosity should not cause blur in the majority of patients.

It has now been found, with consistent use of ophthamological is compositions disclosed herein, visual disturbance can be reduced by improving optical resolution (stability of the tear film), and/or by providing patients with a less viscous product. Also, as measured by contrast sensitivity, visual acuity is improved with the use of said ophthamological compositions.

### SUMMARY OF THE INVENTION

It has now been discovered that novel ophthalmic compositions developed for treating eyes, afflicted or susceptible to diseases/conditions such as, without limitation, dry eye syndrome, low humidity environments, and stress/trauma, for example, due to surgical procedures, and the like, also improve visual acuity. In particular, these compositions would be useful for mitigating the damaging effects of a hypertonic tear film, regardless of cause. The present compositions can be administered, for example, topically administered, to an ocular surface of an eye of a person to prevent the loss of and/or improve visual acuity.

In one broad aspect of the present invention, the ophthalmic compositions comprise a carrier component, advantageously an aqueous carrier component, and an effective amount of a tonicity component including a material selected from compatible solute components, for example, one or more of certain compatible solute agents, and mixtures thereof. In one very useful embodiment, the tonicity component comprises a material selected from erythritol components and mixtures thereof. In one additional embodiment, the tonicity component comprises a material selected from combinations of at least two different compatible solute agents.

In another broad aspect of the invention, ophthalmic compositions for use in the method of the present invention are provided comprising a carrier, for example, an aqueous carrier, component, and an effective amount of a material selected from inositol components, xylitol components and mixtures thereof. The osmolality of such compositions are often higher or greater than isotonic, for example, in a range of at least 310 to about 600 or about 1000 mOsmols/kg.

In a further broad aspect of the invention, ophthalmic compositions for use in the method of the present invention are provided which comprise a carrier, for example, an aqueous carrier, component, and an effective amount of a tonicity component comprising a material selected from carnitine components and mixtures thereof. In a particularly useful embodiment, the composition has a non-isotonic osmolality.

In an additional aspect of the present invention, ophthalmic compositions for use in the method of the present invention are provided which comprise a carrier, for example, an aqueous carrier, component, and an effective amount of a tonicity component comprising a material selected from a mixture or combination of compatible solute agents, for example, selected from mixtures of one or more polyol components and/or one or more amino acid components.

In each of the above-noted aspects of the invention, the present compositions for use in the method of the present invention advantageously have chemical make-ups so as the material or the mixture of organic compatible solute included in the tonicity component is effective, when the composition is administered to an eye, to allow an ocular surface of the eye to better tolerate a hypertonic condition on the ocular surface relative to an identical composition without the material or the mixture of organic compatible solute agents.

A still further broad aspect of the invention provides ophthalmic compositions for use in the method of the present invention comprising carrier component, a tonicity component and a polyanionic component. The tonicity component is present in an amount effective to provide the composition with a desired osmolality, and comprises a compatible solute component. The polyanionic component is present in an amount, when the composition is administered to a human or animal eye, to reduce at least one adverse effect of a cationic, for example, a polycationic, material on an ocular surface of a human or animal eye relative to an identical composition without the polyanionic component. This cationic material could be from any source, for example, may be endogenous, an environmental contaminant, or as an undesired consequence of applying an agent to the eye, for example a preserved solution or contact lens care product. In one very useful embodiment, hyaluronic acid is not the is sole polyanionic component. Other polyanionic components are more suited for use in the present compositions, for example, are more suited than hyaluronic acid or its salts for topical administration to an ocular surface of a human or animal eye. In another embodiment of the present invention, the composition has an osmolality in a range of about 300 to about 600 or about 1000 mOsmols/kg.

One further broad aspect of the invention provides ophthalmic compositions for use in the method of the present invention comprising a carrier component, and a polyanionic component selected from polyanionic peptides, polyanionic peptide analogs, portions of polyanionic peptide analogs, carboxymethyl-substituted polymers of sugars, including but not limited to, glucose and the like sugars and mixtures thereof. Such polyanionic components are present in an amount effective, when the compositions are administered to a human or animal eye, to reduce at least one adverse effect of a cationic, for example, polycationic, species and/or substance on an ocular surface of the eye relative to an identical composition without the polyanionic component.

Any and all features described herein and combinations of such features are included within the scope of the present invention provided that the features of any such combination are not mutually inconsistent.

These and other aspects of the present invention, are apparent in the following detailed description, accompanying drawings, examples and claims.

### BRIEF DESCRIPTION OF THE DRAWINGS

FIG. 1 is a graphical presentation of the intensity with regard to phosphorylated c-jun N-terminal kinases (p-JNK1 and p-JNK2) of certain ophthalmic compositions.

FIG. 2 is a graphical presentation of the intensity with regard to p-JNK1 and p-JNK2 of certain other ophthalmic compositions.

FIG. 3 is a graphical presentation of Phosphorylated:total JNK ratios for certain ophthalmic compositions obtained using the Beadlyte method.

FIG. 4 is a graphical presentation of Phospho:total p38 MAP Kinase for certain ophthalmic compositions obtained using the Beadlyte method.

FIG. 5 is a graphical presentation of Phospho:total ERK MAP Kinase for certain ophthalmic compositions obtained using the Beadlyte method.

FIG. 6 is a graphical presentation of a summary of concentration dependent effects on trans-epithelial electrical resistance (TEER) for various ophthalmic compositions.

FIG. 7 is a graphical presentation of the effects on TEER of various ophthalmic compositions including compositions including combinations of compatible solute agents.

FIG. 8 is a graphical presentation of the effects on TEER of various other ophthalmic compositions including compositions including combinations of compatible solute agents.

FIG. 9 is the OSDI, which is a validated 12-item patient-reported outcomes questionnaire designed to provide an assessment of various symptoms, related visual functions and environmental triggers of dry eye.

FIG. 10 is a breakdown of Subjective Evaluation of Symptom or dryness, i.e., SEoSD normal/dry eye categories according to score.

FIG. 11 shows the baseline and day 30 OSDI scores obtained in two clinical studies evaluating a preserved and preservative-free ophthalmic composition of the invention.

FIG. 12 shows the baseline and 30 SESoD scores obtained in two clinical studies evaluating a preserved and preservative-free ophthalmic composition of the invention.

FIG. 13 shows the baseline and day 30 OSDIᵥ scores obtained in two clinical studies evaluating a preserved and preservative-free ophthalmic composition of the invention.

FIG. 14 shows the dryness and vision VAS scores at baseline and day 30 of a clinical trial that evaluated a preservative-free ophthalmic composition of the invention.

FIG. 15(a)-(c) shows the correlation between OSDI and vision VAS from a clinical trial that evaluated a preservative-free ophthalmic composition of the invention

### DETAILED DESCRIPTION OF THE INVENTION

The present invention provides a method of improving the visual acuity of a person in need thereof which comprises topically administering to said person, in an effective amount, an ophthalmic composition comprising an aqueous carrier component; and an effective amount of a tonicity component comprising a material selected from a combination of compatible solute agents, wherein said combination of compatible solute agents comprises two polyol components and one amino acid component and wherein said polyol components are erythritol and glycerol and said amino acid component is carnitine.

As used herein “improving” means increasing peak acuity (resolution) and/or extending the time period of clear vision at or near peak acuity.)

An “effective amount” is that amount of material which is effective, when administered to an eye, to allow an ocular surface of an eye to better tolerate a hypertonic condition on the ocular surface relative to an identical composition without the material.

Although such compositions may have any suitable tonicity or osmolality, for example, a hypotonic osmolality, a substantially isotonic osmolality or a hypertonic osmolality, very useful compositions have osmolalities other than isotonic osmolality, for example, greater than isotonic osmolality. In one embodiment, the compositions useful in the method of the present invention have osmolalities in a range of at least about 300 or about 310 to about 600 or about 1000 mOsmols/kg.

Polyols, such as erythritol components, xylitol components, inositol components, and the like and mixtures thereof, are effective tonicity/osmotic agents, and may be included, alone or in combination with glycerol and/or other compatible solute agents, in the present compositions. Without wishing to limit the invention to any particular theory of operation, it is believed that because of their increased size, relative to glycerol, these polyol components when used topically on the eye, accumulate in the cells more slowly than glycerol, but remain within the cells for prolonged periods of time relative to glycerol.

In one very useful embodiment, mixtures of two or more different compatible solute components, for example, glycerol and/or one or more other polyol components and/or one or more other compatible solute components, for example, is one or more uncharged or zwitterionic amino acid components and the like, may be advantageously used together to provide one or more benefits to the eye that are not obtained using compositions including only a single compatible solute component.

As used herein, the term “component” as used herein with reference to a given compound refers to the compound itself, isomers and steroisomers, if any, of the compound, suitable salts of the compound, derivatives of the compound and the like and mixtures thereof.

As use herein, the term “derivative” as it relates to a given compound refers to a compound having a chemical make-up or structure sufficiently similar to the given compound so as to function in a manner substantially similar to a substantially identical to the given compound in the present compositions and/or methods.

Comfort and tolerability can be considered in formulating the compositions used in the method of the present invention. The amount of organic compatible solute component employed in the said compositions should be effective in providing at least one benefit to the eye of a patient without unduly adversely affecting the patient, for example, without unduly inducing discomfort, reflex tearing and the like adverse affects.

For a formulator schooled in the art, it is possible to make thick fluids and gels that are retained for greater periods on the ocular surface than thin fluids, with the trade-off often being a transient vision blur. Thick fluids and gels thus have the disadvantage of negatively affecting the improvement in visual acuity in compositions that would otherwise improve the visual acuity of a person in need of improvement.

Xylitol or erythritol used alone may require prolonged contact time to allow them to function effectively as a compatible solute component, for example, due to the time needed for cellular uptake. However once in situ, for example, within ocular surface cells, the beneficial action of balancing hypertonic conditions advantageously is longer than with an equivalent amount of glycerol, which moves more quickly into and out of cells. Such longer lasting benefit, and less frequent dosing, can be obtained without blurred vision.

In one embodiment, the compositions utilized in the present method include a combination or mixture of compatible solute agents, with each agent advantageously is being of different chemical type and/or having a different molecular size and/or mobility. Small mobile agents offer rapid but short duration effectiveness, e.g., protection from hypertonic insult, whereas large less mobile agents offer delayed but longer lasting protection effectiveness.

Xylitol, erythritol and glycerol all have high hydroxyl group concentrations: one per carbon. Hydroxyl groups allow for greater water binding and increase compound solubility. In compositions for treatment of dry eye syndrome, such high hydroxy group concentration may enhance performance of the composition by preventing water loss from the tissues.

Among the polyols, the 5-carbon xylitol, 4-carbon erythritol, and 3-carbon glycerol are preferred for ophthalmic use. The 2-carbon form (ethylene glycol) is a well-known toxin and is not suitable. The 6-carbon forms (mannitol, sorbitol, and related deoxy compounds) may be useful in combination with the smaller molecules. In one embodiment, combinations of polyols with 3 to 6 carbons, and 1 and 2 carbon deoxy derivatives including, without limitation, isomers, stereo-isomers and the like, as appropriate, may be useful in the present invention.

Uncharged or zwitterionic amino acids are useful as organic compatible solute components in accordance with the present invention.

Carnitine components, for example, carnitine itself, isomers/stereo-isomers thereof, salts thereof, derivatives thereof and the like and mixtures thereof, are very useful compatible solute components for use in the present ophthalmic compositions. Carnitine is well-established as necessary for various parts of fatty acid metabolism, so it has a significant role in the metabolism of liver and muscle cells. Carnitine may serve as an energy source for many types of cells, including ocular cells. Carnitine components may have unique properties in multiple roles, for example as osmoprotectants, in fatty acid metabolism, as an antioxidant, in promoting wound healing, as a protein chaperone, and in neuroprotection.

The organic compatible solute component may be advantageously provided in the present compositions by using a combination of such agents or materials of differing size, mobility, and mechanism of action. Small mobile agents, such as is smaller polyols, would be predicted to offer rapid but short duration osmoprotection. Several of the amino acids and related compounds may function as long-acting intracellular compatible solutes and protein stabilizers. In the present invention, carnitine components may be used alone or in combination with one or more other amino organic compatible solute components and/or polyols, for example, as described herein.

Amine-based organic compatible solute components and/or components that may be used include, but are not limited to, betaine, taurine, carnitine, sarcosine, proline, trimethylamines in general, other zwitterionic amino acids and the like and mixtures thereof. Polyols that may be useful in combination with carnitine and/or one of the other amine-based organic compatible solute components include, but are not limited to, glycerol, propylene glycol, erythritol, xylitol, myo-inositol, mannitol, sorbitol and the like and mixtures thereof.

The amount of the compatible solute component included in the compositions utilized in the present method may be any suitable amount. However, such amount advantageously is effective to provide a benefit to the eye as a result of the administration of the composition containing the compatible solute component to the eye. Excessive amounts of compatible solute components are to be avoided, since such amounts can cause discomfort to the patient and/or potential harm to the eye being treated. The compatible solute component advantageously is present in an amount effective in providing the desired osmolality to the composition.

The specific amount of compatible solute component employed may vary over a wide range depending, for example, on the overall chemical make-up and intended use of the composition, on the desired osmolality of the composition, on the specific compatible solute or combination of such solutes being employed and the like factors. In one embodiment, the total amount of compatible solute component included in the present compositions may be in a range of about 0.01% (w/v) or about 0.05% (w/v) to about 1% (w/v) or about 2% (w/v) or about 3% (w/v) or more.

Corneal surface cells respond to osmotic forces by regulating salt and water transport in an effort to maintain a constant cell volume. In conditions of chronic hypertonicity, for example, such as exist in dry eye disease, transport mechanisms for uptake of compatible solutes, including various amino acids and polyols, are up-regulated. In one embodiment of the present invention, ophthalmic compositions, for example, artificial tears, containing a compatible solute component are formulated to have a tonicity higher or in excess of isotonicity, advantageously in a tonicity range of about 300 or about 310 to about 600 or about 1000 mOsmols/kg. Without wishing to limit the invention to any particular theory of operation, it is believed that, under such conditions, both immediate and long-term mechanisms to accumulate compatible solutes in cells are stimulated, allowing enhanced uptake and retention compared to cellular activity under isotonic or hypotonic conditions. Once the compatible solute component is accumulated by the cells, the cells have enhanced protection from ongoing hypertonic insult, for example, caused by dry eye syndrome and/or one or more other conditions/diseases. Results of this enhanced protection include improved cellular metabolism and survival for a period of hours to days following application of an ophthalmic composition of the present invention.

In the normal lacrimal system, tear production, tear drainage, and tear evaporation is balanced in order to provide a moist, lubricated ocular surface. Typical values for tear osmolarity range from 290 to 310 mOsmols/kg in normal individuals, and these may change throughout the day or in response to changing environmental conditions. In the normal individual, neural feedback from the ocular surface to the lacrimal glands controls tear production in order to maintain a stable ocular surface fluid. It has been proposed that tear film tonicity is one of the principal stimuli for this regulatory feedback. In dry eye disease, dysfunction of the production apparatus (the various glands), the drainage system, the neural signaling mechanism, or the ocular surface itself leads to an inadequate tear film, ocular surface compromise, and subjective discomfort.

On the cellular level, dry eye disease is usually characterized by a chronically hypertonic extracellular (tear film) environment. Published reports of the tonicity of the tear film of dry eye patients gives a range of 300 to 500 mOsmols/kg, with most values between 320 and 400 mOsmols/kg. Under these conditions, cells will tend to lose water and/or gain salts, and may undergo cell volume changes. Hypertonicity has been shown to alter cellular metabolic processes, reduce the functioning of enzymatic processes, and lead to apoptosis and cell death.

As a defense against hypertonic challenge, corneal cells have been is demonstrated to up-regulate transport mechanisms for non-ionic solutes such as amino acids and polyols, and accumulate these solutes intracellularly in order to maintain cell volume without changing electrolyte balance. Under these conditions, cellular metabolism is less affected than with volume and electrolyte changes, and such compounds are referred to as compatible solutes. Compatible solutes include but are not limited to the amino acids betaine (trimethylglycine), taurine, glycine, and proline, and the polyols glycerol, erythritol, xylitol, sorbitol, and mannitol. Compatible solutes are also considered to be osmoprotectants since they may allow cell metabolism or enhance cell survival under hypertonic conditions that would otherwise be restricting.

Cells accumulate certain compatible solutes by biosynthesis within the cell and others by increased trans-membrane transport from the extracellular fluid (in this case the tear fluid). In both cases, specific synthetic or transport proteins are involved in this process. Experimental evidence indicates that these proteins are activated in the presence of hypertonic conditions, and that transcription and translation events to produce these proteins are up-regulated by hypertonic conditions. Conversely, experimental evidence indicates that corneal and other cells will expel compatible solutes when exposed to hypotonic conditions, or when moving from a hypertonic to an isotonic environment.

In dry eye disease, corneal surface cells are exposed to a hypertonic environment, and are stimulated to accumulate osmoprotectant substances as they are available. The addition of an iso- or hypo-tonic artificial tear to the ocular surface provides relief from symptoms due to enhanced lubrication, but tends to down-regulate mechanisms in these cells for accumulation of osmoprotectants. This may result in further vulnerability to osmotic insult in the minutes to hours following drop use as the tear film returns to its hypertonic dry eye state.

Current FDA guidance stipulates that “an ophthalmic solution should have an osmotic equivalence between 0.8 and 1.0 percent sodium chloride to comply with labeling claims of ‘isotonic solution’.” This is equivalent to a range from 274 to 342 mOsm/kg. Further, FDA guidelines state that “two to 5 percent sodium chloride ophthalmic preparations are hypertonic and are acceptable OTC products when is labeled as ‘hypertonic solutions’.” This range equates to 684 to 1711 mOsm/kg. For the purposes of the present invention, a “supra-tonic” solution is defined to have an osmolality intermediate between these two ranges, or approximately 300 or 310 to about 600 or about 800 or about 1000 mOsmols/kg, equivalent to about 0.9 to about 1.8 percent sodium chloride (1.8% is the maximum FDA guidance for topical ophthalmic solutions not labeled as hypertonic).

The present invention takes these concepts into account by formulating an artificial tear at supra-tonic levels more compatible with the existing hypertonic state of the dry eye ocular surface. In addition to being formulated in the supra-tonic range (about 300 or about 310 to about 600 or about 1000 mOsmols/kg total tonicity), the present compositions contain one or more organic compatible solute agents as described herein. The combination of supra-tonicity and inclusion of one or more compatible solutes in the present compositions serve to both stimulate or maintain uptake of these protective substances into the corneal surface cells, and to provide abundant supplies of these materials or substances.

In addition to sufficient quantities of compatible solutes in a supra-tonic medium, the present compositions also may contain appropriate demulcents and viscosity agents, which provide comfort and lubrication, and also advantageously are effective in holding the organic compatible solute composition on the ocular surface for sufficient time to enhance uptake by the corneal surface cells.

It should be noted that FDA guidelines clearly indicate that the final tonicity of the formulation may be determined by nonionic as well as ionic species. Thus, the formula may contain significant amounts of glycerol and other compatible solutes, and not contain substantial amounts or any of ionic tonicity agents, such as sodium salts. In one embodiment, the present components are substantially free of ionic tonicity agents.

Advantageously, the present compositions include a combination of different organic compatible solute agents effective to provide for uptake by corneal cells during the time of exposure to the drop during use, for example, about 5 to about 30 minutes, depending on viscosity, after administration, and to provide for intracellular retention during the period of hours between drop applications.

Because of the enhanced protection from osmotic insult provided by the is present composition, the duration of clinical benefit resulting from each dosage or application is increased. With regular use of the present compositions, ocular surface health is enhanced as cells are less metabolically challenged and cell survival is enhanced.

In one useful embodiment of the present method, compositions comprising polyanionic components, for example, with or without the compatible solute components, may be effectively used before, during and/or after surgical procedures, including without limitation, surgical procedures in which the eye is exposed to laser energy, for example, in the treatment of post-LASIK staining, dryness and other ocular surface complications. The etiology of post-LASIK surface compromise may be multifactorial, including, without limitation, surgically-induced neurotrophic hypesthesia and keratitis, damage to limbal cells from force of the suction ring, altered lid apposition in blinking due to altered corneal topography, chemical damage to ocular surface from topical medications and preservatives and the like.

The administration of polylanionic component-containing compositions, in accordance with the present invention, to the ocular surface and tear film may be effective in treating one or more or even all, of the above named causes of post-LASIK ocular surface compromise.

In one particularly useful embodiment, the compositions include polyanionic components that mimic the activity, for example, the anigenic and/or cytotoxic activity, of the pro-piece of MBP, which has been shown to consist of a 90-residue polypeptide. Useful agents may include one or more polypeptide analogs of this sequence or portions of this sequence.

As used herein, the term “mimic” means that the polyanionic component, e.g., polypeptide analog, has an activity within (plus or minus) about 5% or about 10% or about 15% or about 20% of the corresponding activity of the pro-piece of MBP.

The pro-piece of MBP has an amino acid sequence as shown in SEQ ID NO:1 below:

LHLRSETSTF ETPLGAKTLP EDEETPEQEM EETPCRELEE
EEEWGSGSED ASKKDGAVES ISVPDMVDKN LTCPEEEDTV
KVVGIPGCQ

A polypeptide analog of the Major Basic Protein pro-piece sequence or of a portion of the Major Basic Protein pro-piece sequence means a peptide comprising an amino acid sequence having at least about 75% or about 80% or about 85% or about 90% or about 95% or about 99% or more identity to a homologous continuous amino acid sequence comprised in SEQ ID NO:1, or portions thereof.

Carboxymethyl-substituted polymers of sugars, for example and without limitation, glucose and the like sugars, may be employed as polyanionic components in accordance with the present invention.

Further, additional useful polyanionic components include, without limitation, modified carbohydrates, other polyanionic polymers, for example, and without limitation, those already available for pharmaceutical use, and mixtures thereof. Mixtures of one or more of the above-noted polypeptide analogs and one or more of the above-noted other polyanionic components may be employed.

The compositions are advantageously ophthalmically acceptable, comprising an ophthalmically acceptable carrier component, a compatible solute component and/or a polyanionic component.

A composition, carrier component or other component or material is “ophthalmically acceptable” when it is compatible with ocular tissue, that is, it does not cause significant or undue detrimental effects when brought into contact with ocular tissue. Preferably, the ophthalmically acceptable component or material is also compatible with other components of the present compositions.

As used herein, the term “polyanionic component” refers to a chemical entity, for example, an ionically charged species, such as an ionically charged polymeric material, which includes more than one discrete anionic charge, that is multiple discrete anionic charges. Preferably, the polyanionic component is selected from the group consisting of polymeric materials having multiple anionic charges and mixtures thereof.

The polyanionic component may have a substantially constant or uniform molecular weight, or may be made up of two or more polyanionic component portions of different molecular weights. Ophthalmic compositions having polyanionic components including two or more portions of different molecular weights are disclosed in U.S. patent application Ser. No. 10/017,817, filed Dec. 14, 2001, the disclosure of which is hereby incorporated in its entirety herein by reference.

Preferably, the composition has an increased ability to adhere to an eye when the composition is administered to an eye relative to a substantially identical composition without the polyanionic component. With regard to the increased ability to adhere to an eye feature noted above, the present compositions preferably are effective to provide effective lubrication over a longer period of time before requiring readministration relative to a substantially identical composition without the polyanionic component.

Any suitable polyanionic component may be employed in accordance with the present invention provided that it functions as described herein and has no substantial detrimental effect on the composition as a whole or on the eye to which the composition is administered. The polyanionic component is preferably ophthalmically acceptable at the concentrations used. The polyanionic component preferably includes three (3) or more anionic (or negative) charges. In the event that the polyanionic component is a polymeric material, it is preferred that many of the repeating units of the polymeric material include a discrete anionic charge. Particularly useful anionic components are those which are water soluble, for example, soluble at the concentrations used in the present compositions at ambient (room) temperature.

Examples of suitable polyanionic components useful in the present compositions include, without limitation, anionic cellulose derivatives, anionic acrylic acid-containing polymers, anionic methacrylic acid-containing polymers, anionic amino acid-containing polymers and mixtures thereof. Anionic cellulose derivatives are very useful in the present invention.

A particularly useful class of polyanionic components are one or more polymeric materials having multiple anionic charges. Examples include, but are not is limited to:

metal carboxy methylcelluloses

metal carboxy methyl hydroxyethylcelluloses

metal carboxy methylstarchs

metal carboxy methylhydroxyethylstarchs

metal carboxy methylpropyl guars

hydrolyzed polyacrylamides and polyacrylonitriles

heparin

gucoaminoglycans

hyaluronic acid

chondroitin sulfate

dermatan sulfate

peptides and polypeptides

alginic acid

metal alginates

homopolymers and copolymers of one or more of:
acrylic and methacrylic acids
metal acrylates and methacrylates
vinylsulfonic acid
metal vinylsulfonate
amino acids, such as aspartic acid, glutamic acid and the like
metal salts of amino acids
p-styrenesulfonic acid
metal p-styrenesulfonate
2-methacryloyloxyethylsulfonic acids
metal 2-methacryloyloxethylsulfonates
3-methacryloyloxy-2-hydroxypropylsulonic acids
metal 3-methacryloyloxy-2-hydroxypropylsulfonates
2-acrylamido-2-methylpropanesulfonic acids
metal 2-acrylamido-2-methylpropanesulfonates
allylsulfonic acid
metal allylsulfonate and the like.

is Excellent results are achieved using polyanionic components selected from carboxy methylcelluloses and mixtures thereof, for example, alkali metal and/or alkaline earth metal carboxy methylcelluloses.

The present compositions preferably are solutions, although other forms, such as ointments, gels, and the like, may be employed.

The carrier component is ophthalmically acceptable and may include one or more components which are effective in providing such ophthalmic acceptability and/or otherwise benefiting the composition and/or the eye to which the composition is administered and/or the patient whose eye is being treated. Advantageously, the carrier component is aqueous-based, for example, comprising a major amount that is at least about 50% by weight, of water. Other components which may be included in the carrier components include, without limitation, buffer components, tonicity components, preservative components, pH adjustors, components commonly found in artificial tears and the like and mixtures thereof.

The compositions preferably have viscosities in excess of the viscosity of water. In one embodiment, the viscosity of the present compositions is at least about 10 cps (centipoise), more preferably in a range of about 10 cps to about 500 cps or about 1,000 cps. Advantageously, the viscosity of the present composition is in a range of about 15 cps or about 30 cps or about 70 to about 150 cps or about 200 cps or about 300 cps or about 500 cps. The viscosity of the present composition may be measured in any suitable, for example, conventional manner. A conventional Brookfield viscometer measures such viscosities.

In one very useful embodiment, the polyanionic component is present in an amount in a range of about 0.1% to about 5%, preferably about 0.2% to about 2.5%, more preferably about 0.2% to about 1.8% and still more preferably about 0.4% to about 1.3% (w/v) of the composition.

Other components which may be included in the carrier components include, without limitation, buffer components, tonicity components, preservative-components, pH adjustors, components commonly found in artificial tears, such as one or more electrolytes, and the like and mixtures thereof. In one very useful embodiment the carrier component includes at least one of the following: an effective amount of a buffer component; an effective amount of a tonicity component; an effective amount of is a preservative component; and water.

These additional components preferably are ophthalmically acceptable and can be chosen from materials which are conventionally employed in ophthalmic compositions, for example, compositions used to treat eyes afflicted with dry eye syndrome, artificial tear formulations and the like.

Acceptable effective concentrations for these additional components in the compositions of the invention are readily apparent to the skilled practitioner.

The carrier component preferably includes an effective amount of a tonicity adjusting component to provide the composition with the desired tonicity. The carrier component preferably includes a buffer component which is present in an amount effective to maintain the pH of the composition in the desired range. Among the suitable tonicity adjusting components that may be employed are those conventionally used in ophthalmic compositions, such as one or more various inorganic salts and the like. Sodium chloride, potassium chloride, mannitol, dextrose, glycerin, propylene glycol and the like and mixtures thereof are very useful tonicity adjusting components. Among the suitable buffer components or buffering agents that may be employed are those conventionally used in ophthalmic compositions. The buffer salts include alkali metal, alkaline earth metal and/or ammonium salts, as well as citrate, phosphate, borate, lactate and the like salts and mixtures thereof. Conventional organic buffers, such as Goode's buffer and the like, may also be employed.

Any suitable preservative component may be included in the present compositions provided that such components are effective as a preservative in the presence of the polyanionic component. Thus, it is important that the preservative component be substantially unaffected by the presence of the polyanionic component. Of course, the preservative component chosen depends on various factors, for example, the specific polyanionic component present, the other components present in the composition, etc. Examples of the useful preservative components include, but are not limited to, per-salts, such as perborates, percarbonates and the like; peroxides, such as very low concentrations, e.g., about 50 to about 200 ppm (w/v), of hydrogen peroxide and the like; alcohols, such as benzyl alcohol, chlorbutanol and like; sorbic acid and ophthalmically acceptable salts thereof and mixtures thereof.

The amount of preservative component included in the present compositions containing such a component varies over a relatively wide range depending, for example, on the specific preservative component employed. The amount of such component preferably is in the range of about 0.000001% to about 0.05% or more (w/v) of the present composition.

One particularly useful class of preservative components are chlorine dioxide precursors. Specific examples of chlorine dioxide precursors include stabilized chlorine dioxide (SCD), metal chlorites, such as alkali metal and alkaline earth metal chlorites, and the like and mixtures thereof. Technical grade sodium chlorite is a very useful chlorine dioxide precursor. Chlorine dioxide-containing complexes, such as complexes of chlorine dioxide with carbonate, chlorine dioxide with bicarbonate and mixtures thereof are also included as chlorine dioxide precursors. The exact chemical composition of many chlorine dioxide precursors, for example, SCD and the chlorine dioxide complexes, is not completely understood. The manufacture or production of certain chlorine dioxide precursors is described in McNicholas U.S. Pat. No. 3,278,447, which is incorporated in its entirety herein by reference. Specific examples of useful SCD products include that sold under the trademark Purite 7 by Allergan, Inc., that sold under the trademark Dura Klor by Rio Linda Chemical Company, Inc., and that sold under the trademark Anthium Dioxide by International Dioxide, Inc.

The chlorine dioxide precursor is included in the present compositions to effectively preserve the compositions. Such effective preserving concentrations preferably are in the range of about 0.0002 or about 0.002 to about 0.02% (^{w}/ᵥ) or higher of the present compositions.

In the event that chlorine dioxide precursors are employed as preservative components, the compositions preferably have an osmolality of at least about 200 mOsmol/kg and are buffered to maintain the pH within an acceptable physiological range, for example, a range of about 6 to about 8 or about 10.

The compositions preferably include an effective amount of an electrolyte component, that is one or more electrolytes, for example, such as is found in natural tears and artificial tear formulations. Examples of particularly useful such electrolytes for inclusion in the present compositions include, without limitation, alkaline earth is metal salts, such as alkaline earth metal inorganic salts, and mixtures thereof, e.g., calcium salts, magnesium salts and mixtures thereof. Very good results are obtained using an electrolyte component selected from calcium chloride, magnesium chloride and mixtures thereof.

The amount or concentration of such electrolyte component in the present compositions can vary widely and depends on various factors, for example, the specific electrolyte component being employed, the specific composition in which the electrolyte is to be included and the like factors. In one useful embodiment, the amount of the electrolyte component is chosen to at least partially resemble, or even substantially resemble, the electrolyte concentration in natural human tears. Preferably, the concentration of the electrolyte component is in the range of about 0.01 to about 0.5 or about 1% of the present composition.

The compositions may be prepared using conventional procedures and techniques. For example, the present compositions can be prepared by blending the components together, such as in one bulk.

To illustrate, in one embodiment, the polyanionic component portions are combined with purified water and caused to disperse in the purified water, for example, by mixing and/or agitation. The other components, such as the buffer component, tonicity component, electrolyte component, preservative component and the like, are introduced as the mixing continues. The final mixture is sterilized, such as steam sterilized, for example, at temperatures of at least about 100° C., such as in a range of about 120° C. to about 130° C., for a time of at least about 15 minutes or at least about 30 minutes, such as in a range of about 45 to about 60 minutes. In one embodiment, the preservative component preferably is added to the mixture after sterilization. The final product preferably is filtered, for example, through a 20 micron sterilized cartridge filter, such as a 20 micron clarity filter cartridge, e.g., sold by Pall under the tradename HDC II, to provide a clear, smooth solution, which is then aseptically filled into containers, for example, low density polyethylene teal containers.

Alternately, each of the polyanionic component portions can be mixed with purified water to obtain individual polyanionic component portion solutions. By mixing the individual polyanionic component portion solutions together, a blend is easily and effectively obtained having the desired, controlled ratio of the individual polyanionic component portions. The blended solution can then be combined with the other components, sterilized and filled into containers, as noted above.

In one particularly useful embodiment, a solution of the polyanionic component portions and purified water is obtained, as noted above. This solution is then sterilized, for example, as noted above. Separately, the other components to be included in the final composition are solubilized in purified water. This latter solution is sterile filtered, for example, through a 0.2 micron sterilizing filter, such as that sold by Pall under the tradename Suporflow, into the polyanionic component-containing solution to form the final solution. The final solution is filtered, for example, as noted above, to provide a clear, smooth solution which is then aseptically filled into containers, as noted above.

The compositions may be effectively used, as needed, by methods which comprise administering an effective amount of the composition to an eye in need of lubrication, for example, an eye afflicted with dry eye syndrome or having a propensity toward dry eye syndrome. The administering step may be repeated as needed to provide effective lubrication to such eye. The mode of administration of the present composition depends on the form of the composition. For example, if the composition is a solution, drops of the composition may be applied to the eye, e.g., from a conventional eye dropper. In general, the present compositions may be applied to the surface of the eye in substantially the same way as conventional ophthalmic compositions are applied. Such administration of the present compositions does provide substantial and unexpected benefits, as described elsewhere herein.

The following non-limiting examples illustrate certain aspects of the present invention.

### Example 1

In this experiment, corneal epithelial cells were isolated from the rabbit eye and grown under conditions so that they differentiate into a layered “air-lift” culture that includes basal, wing, and squamous cells. As they grow and differentiate, these cultures developed tight junctions between cells that provide the basis for a trans-epithelial electrical resistance (TEER) across the cell layers between the apical and basal surfaces. The TEER value is a sensitive measure of cell growth, differentiation and health.

After 5 days in culture during which the layered structure forms, different culture wells were exposed to hypertonic fluid (400 mOsmols/kg) with or without addition of one of 6 candidate compatible solutes at a low concentration (2 mM). The TEER was then measured after 22 hours of exposure. The TEER value was expressed as a percentage of the TEER value obtained from a similar culture under isotonic (300 mOsmol/kg) conditions. The results of these tests are shown in Table 1.

TABLE 1
Test Results

| | TEER (as % of isotonic |
|---|---|
| Compatible Solute | control) at 22 hours |
| Isotonic Control | 100% |
| Hypertonic Control | 23.3 |
| 2 mM Taurine | 39.8 |
| 2 mM Betaine | 53.3 |
| 2 mM Carnitine | 118.9 |
| 2 mM Erythritol | 107.4 |
| 2 mM Myo-Inositol | 74.8 |
| 2 mM Xylitol | 94.1 |

These results demonstrate that all of the candidates tested have some osmoprotective ability, increasing the TEER relative to the hypertonic control. Surprisingly, of the agents tested, carnitine produced the most benefit. Without wishing to limit the invention to any particular theory of operation, it is believed that the beneficial results obtained with carnitine may relate to carnitine's multiple roles in energy metabolism and other cellular mechanisms as well as its osmoprotective effects.

Further, and also unexpectedly, erythritol provided the best results among the polyols tested. Xylitol and myo-inositol provided good results.

These results indicate that each of the 6 candidate compounds, and preferably, carnitine, erythritol, xylitol and myo-inositol, may be useful in ophthalmic compositions, for example, to mitigate against hypertonic conditions on ocular surfaces of human or animal eyes.

Again, without wishing to limit the invention to any particular theory of operation, it is believed that, due to the varying roles a number of these compounds may play, that combinations of 2 or more of these compounds, for example, including at least one polyol and at least one amino acid, are likely to provide increased protection of corneal surfaces from insults, for example, due to desiccation and hyperosmolality, such as occur in dry eye disease.

### Example 2

Phosphorylated JNK (the activated form of the stress associated protein kinase, SAPK) plays a key role in induction of inflammation and apoptosis in response to stress, including hyperosmolarity.

Human corneoscleral tissues, from donors aged 16-59 years were obtained from the Lions Eye Bank of Texas (Houston, Tex.). Corneal epithelial cells were grown from limbal explants. In brief, after carefully removing the central cornea, excess conjunctiva and iris and corneal endothelium, the limbal rim was cut into 12 equal pieces (about 2×2 mm size each). Two of these pieces were placed epithelial side up into each well of 6-well culture plates, and each explant was covered with a drop of fetal bovine serum (FBS) overnight. The explants were then cultured in SHEM medium, which was an 1:1 mixture of Dulbecco modified Eagle medium (DMEM) and Ham F-12 medium containing 5 ng/mL EGF, 5 μg/mL insulin, 5 μg/mL transferrin, 5 ng/mL sodium selenite, 0.5 μg/mL hydrocortisone, 30 ng/mL cholera toxin A, 0.5% DMSO, 50 μg/mL gentamicin, 1.25 μg/mL amphotericin B and 5% FBS, at 37° C. under 5% CO₂ and 95% humidity. The medium was renewed every 2-3 days. Epithelial phenotype of these cultures was confirmed by characteristic morphology and immuno-fluorescent staining with cytokeratin antibodies (AE-1/AE-3).

Cell culture dishes, plates, centrifuge tubes and other plastic ware were purchased from Becton Dickinson (Lincoln Park, N.J.). Dulbecco modified Eagle medium (DMEM), Ham F-12 medium, Fungizone, and gentamicin were from Invitrogen-GIBCO BRL (Grand Island, N.Y.). Fetal bovine serum (FBS) was from Hyclone (Logan, Utah).

A series of primary sub-confluent corneal epithelial cultures (grown for 12 to 14 days, about 4-5×10⁵ cells/well) were washed three times with preserved buffered saline (PBS) and switched to an Earle's Balanced Salt Solution (EBSS, 300 mOsmols/kg) for 24 hours before treatment. The corneal epithelial cells were cultured for 1 hour in an equal volume (2.0 mL/well) of EBSS media or 400 mOsmols/kg media by adding 53 mM NaCl or sucrose, with either L-carnitine inner salt, betaine hydrochloride, erythritol, or xylitol (all at a concentration of 2 mM) that were pre-added 60 minutes before adding NaCl or sucrose. Samples without these osmoprotectants were also prepared and tested.

The adherent cells were lysed in Beadlyte® Buffer B (included in the Beadlyte® Cell Signaling buffer kit, Upstate Biotechnology, Lake Placid, N.Y.) containing an EDTA-free protease inhibitor cocktail tablet (Roche Applied Science, Indianapolis, Ind.) for 15 minutes. The cell extracts were centrifuged at 12,000×g for 15 minutes at room temperature and the supernatants were stored at −80° C. until they were analyzed by Western blot analysis. The total protein concentrations of the cell extracts were determined using a Micro BCA protein assay kit (Pierce, Rockford, Ill.).

The intensity of each of JNK1 and JNK2 was tested for each of these compositions using Western blot analysis with specific antibodies to each phosphorylated species.

The Western blot analysis was conducted as follows. The protein samples (50 μg per lane) were mixed with 6×SDS reducing sample buffer and boiled for 5 minutes before loading. Proteins were separated by SDS polyacrylamide gel electrophoresis (4-15% Tris-HCl, gradient gels from Bio-Rad, Hercules, Calif.), and transferred electronically to polyvinylidine difluoride (PVDF) membranes (Millipore, Bedford, Mass.). The membranes were blocked with 5% non-fat milk in TTBS (50 mM Tris, pH 7.5, 0.9% NaCl, and 0.1% Tween-20) for 1 hour at room temperature (RT), and then incubated 2 hours at RT with a 1:1000 dilution of rabbit antibody against phospho-p38 MAPK (Cell Signaling, Beverly, Mass.), 1:100 dilution of rabbit antibody against phospho-JNK, or 1:500 dilution of monoclonal antibody against phospho-p44/42 ERK (Santa Cruz Biotechnology, Santa Cruz, Calif.).

After three washings with TTBS, the membranes were incubated for 1 hour at RT with horseradish peroxidase-conjugated secondary antibody goat anti-rabbit IgG (1:2000 dilution, Cell Signaling, Beverly, Mass.), or goat anti-mouse IgG (1:5000 dilution, Pierce, Rockford, Ill.). After washing the membranes four times, the signals were detected with an ECL advance chemiluminescence reagent (Amersham, Piscataway, N.J.) and the images were acquired by a Kodak image station 2000R (Eastman Kodak, New Haven, Conn.). The membranes were stripped in 62.5 mM Tris HCl, pH 6.8, containing 2% SDS and 100 mM α-mercaptoethanol at 60° C. for 30 minutes, then they were re-probed with 1:100 dilution of rabbit antibody against JNK (Santa Cruz Biotechnology) or 1:1000 dilution of rabbit antibodies against ERK or p38 MAPK (Cell Signaling). These three antibodies detect both phosphorylated and un-phosphorylated forms which represent the total levels of these MAPKs. The signals were detected and captured as described above.

An intensity score is determined from image analysis of the resulting bands.

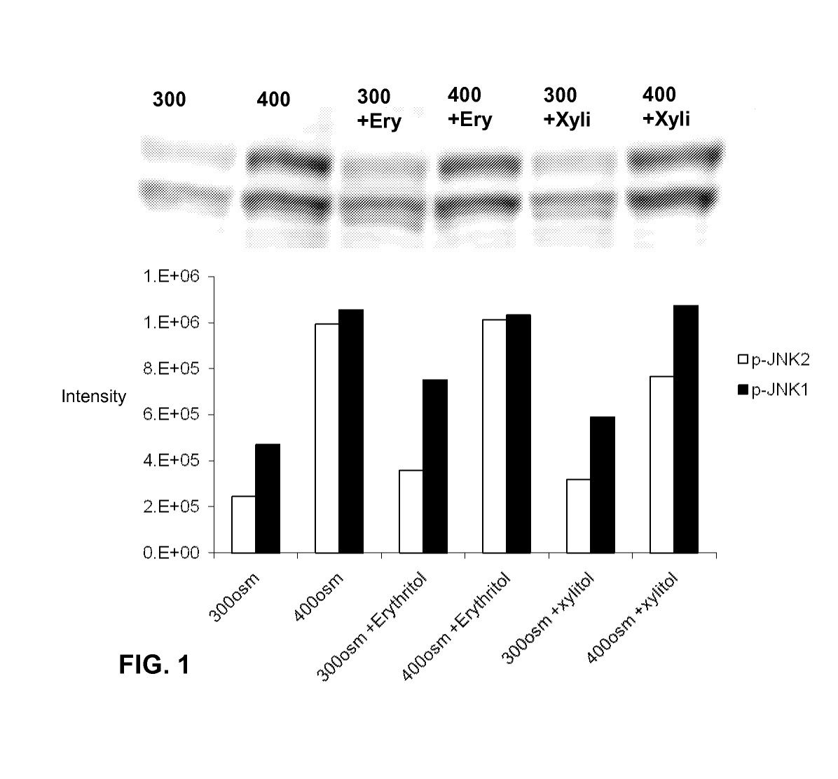

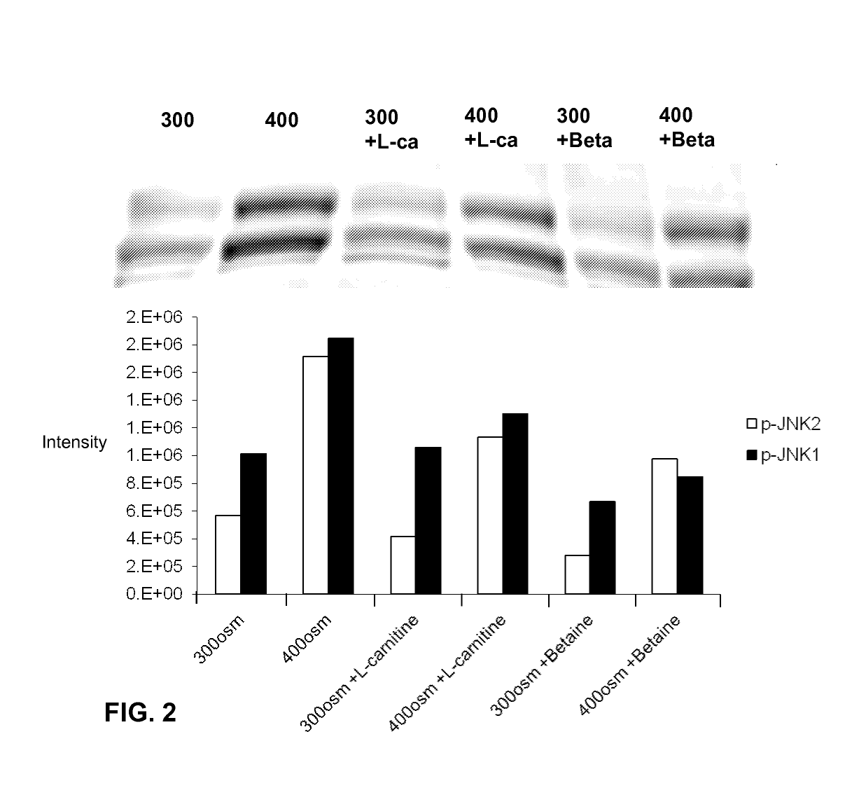

Test results are shown in FIGS. 1 and 2.

Referring now to FIG. 1, there was no effect on JNK activation with either erythritol or xylitol. However, with reference to FIG. 2, there was a definite decrease in the levels of JNK1 and JNK2 mL-carnitine and betaine cultures compared to 400 mOsmols/kg media alone. There was also a less robust effect in the 300 mOsmols/kg cultures.

### Example 3

In another series of experiments, the Beadlyte® Cell Signaling Assay was used. This assay is a fluorescent bead-based sandwich immunoassay. Each sample (10 μg/25 μL) was pipetted into a well of a 96-well plate and incubated with 25 μL of diluted 5× beads coupled to phospho-JNK, phospho-ERK, phospho-p38 or total JNK, or total ERK, or total p38 specific capture antibodies overnight. Overnight incubation was utilized for the reaction of the capture beads with the proteins from the cell lysates.

The beads were washed and mixed with biotinylated specific reporter antibodies for phospho-MAPK or total-MAPK, followed by streptavidin-phycoerythrin. The amount of total or phospho-MAPK was then quantified by the Luminex 100™ system (Luminex, Austin, Tex.). Fifty events per bead were read, and the data output obtained from the Bio-Plex Manager software were exported to Microsoft Excel® for further analysis. The results were presented as the percentage of phospho-MAPK to total-MAPK.

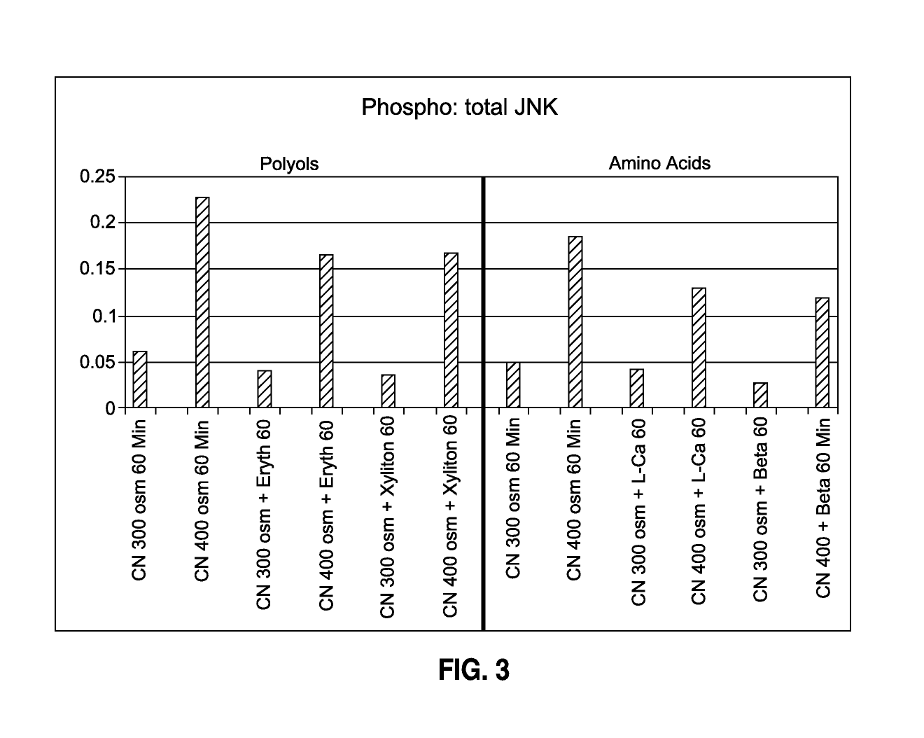

Results of these tests are shown in FIGS. 3, 4 and 5.

As shown in FIG. 3, all of the candidate materials, that is, all of erythritol, xylitol, L-carnitine and betaine, reduced the amount of phospho-total JNK relative to the hypertonic control.

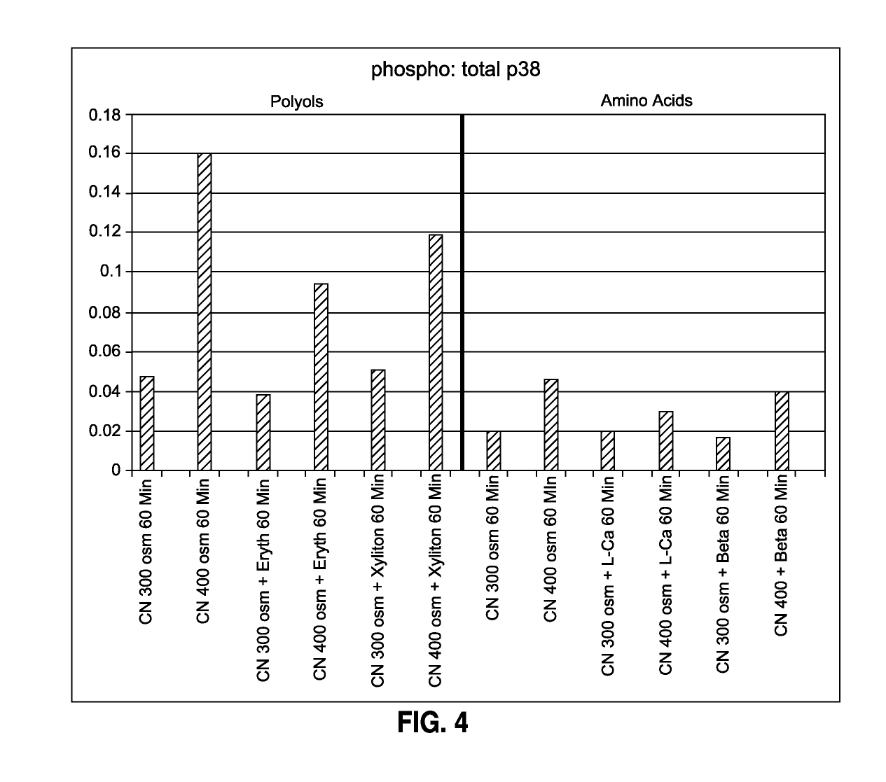

With reference to FIG. 4, all of the candidate materials, with the exception of betaine, reduced the amount of phospho-total p 38 relative to the hypertonic control.

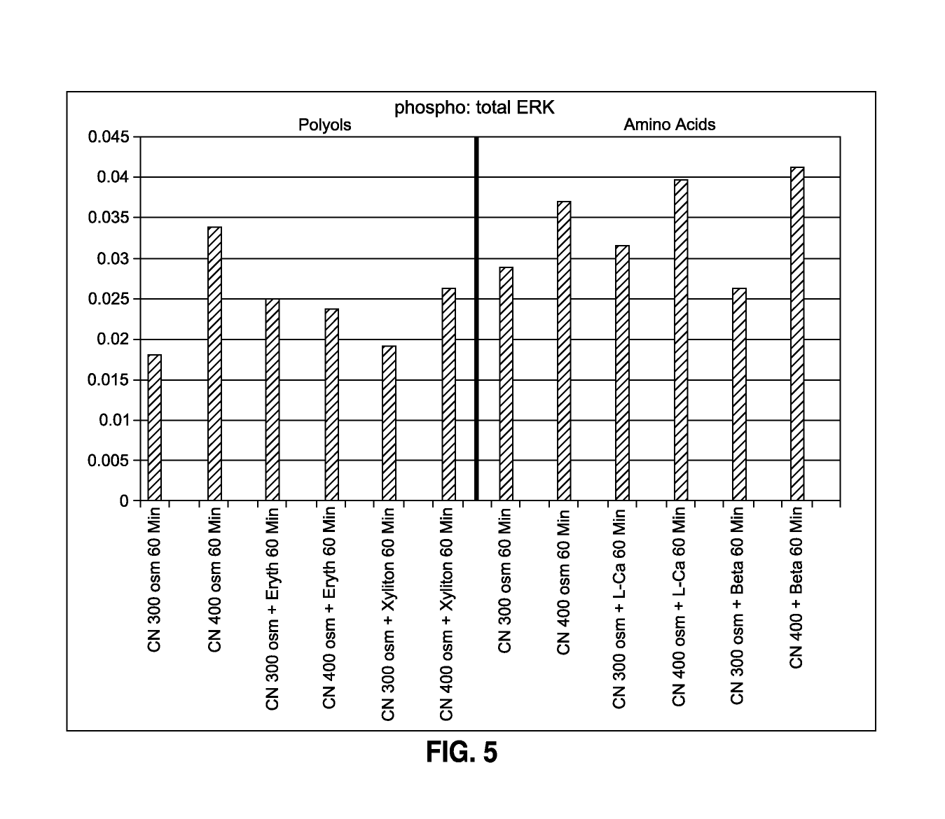

As shown in FIG. 5, the polyol candidate materials, that is erythritol and xylitol reduced the amount of ERK relative to the hypertonic control. The amino acids, betaine and carnitine did not.

### Example 4

Example 1 is repeated except that different concentrations of each of the candidate materials are used, and the TEER is measured at various times from 0 to 24 hours.

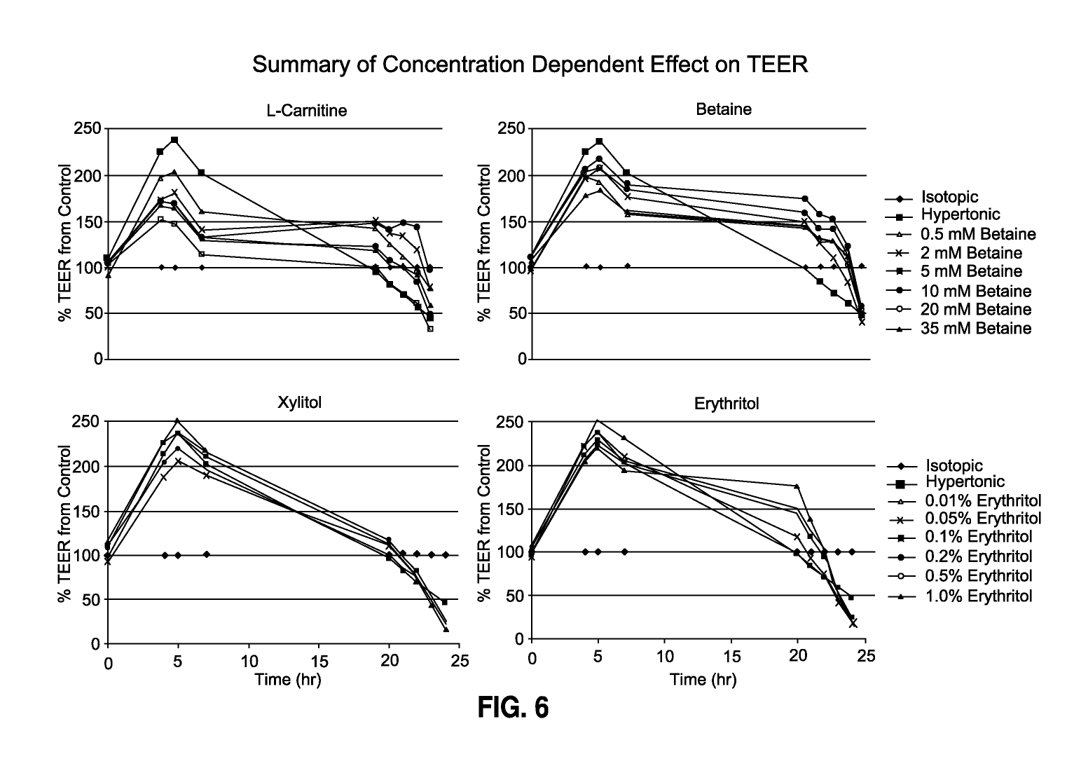

Results of these tests are shown in FIG. 6. As in Example 1, the TEER variable is represented as % TEER relative to the isotonic control.

These results demonstrate that a dose-related response was observed for L-carnitine, betaine and erythritol.

A composition including betaine and stabilized chlorine dioxide, as a preservative, was tested for component compatibility. It was found that the betaine was not fully compatible in such a composition. Thus, betaine is not useful with is certain preservatives, such as stabilized chlorine dioxide. However, betaine may advantageously be employed as a compatible solute in ophthalmic compositions which use other preservative systems, or which are free of preservatives, for example, in single or unit-dose applications.

### Example 5

Example 4 was repeated except that compositions including combinations of compatible solutes were used. Compositions including only glycerol as a compatible solute were also tested.

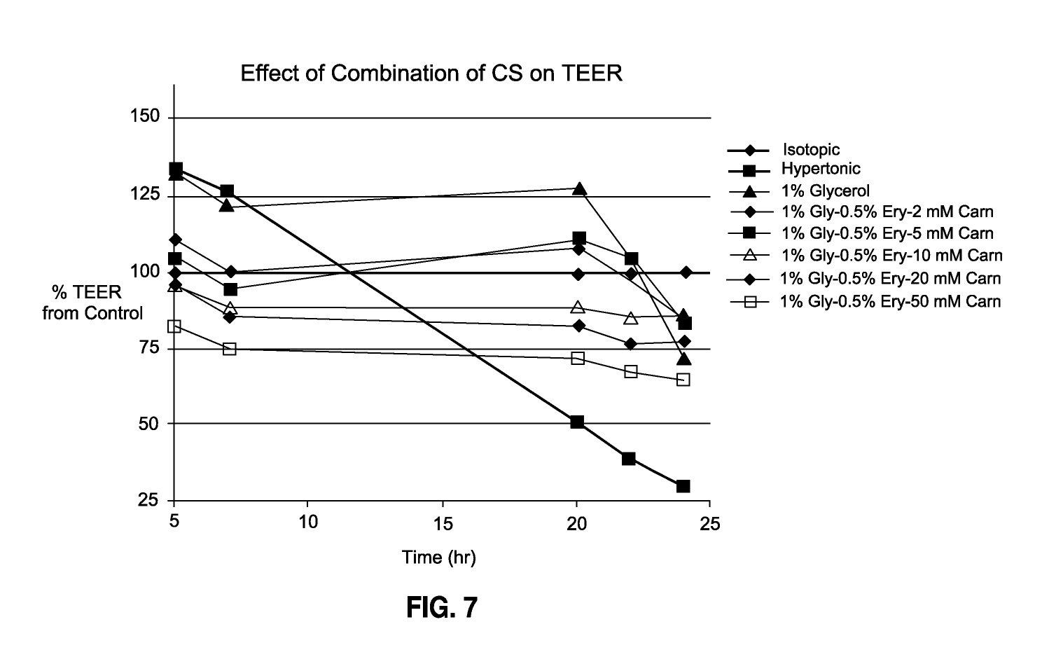

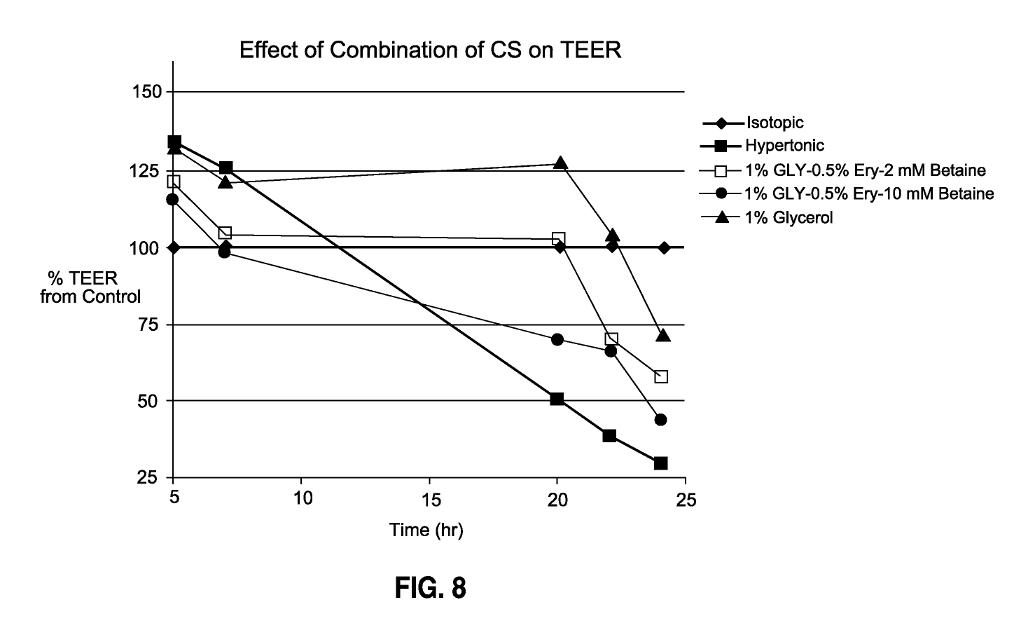

Test results are shown in FIGS. 7 and 8.

These test results demonstrate that combinations of different compatible solutes may potentially yield added benefits.

### Example 6

The pro-piece of Major Basic Protein (MBP) has been shown to be a 90-residue polypeptide.

Using established and well known techniques, a polypeptide analog of the sequence of this 90-residue polypeptide is produced.

An ophthalmic composition is prepared by blending together the following components:

| | Concentration |
|---|---|
| Above-noted | % (w/v) |
| Polypeptide analog | 0.5% |
| Glycerol | 1.0% |
| Erythritol | 0.5% |
| Boric Acid | 0.65 |
| Sodium Borate | 0.25 |
| Sodium Citrate | 0.1 |
| Potassium Chloride | 0.01 |
| Purite ®⁽¹⁾ | 0.01 |
| Sodium Hydroxide 1N | Adjust pH to 7.2 |
| Hydrochloride acid 1N | Adjust pH to 7.2 |
| Purified Water | q.s. ad. |

⁽¹⁾Purite7 is a registered trademark of Allergan, Inc. for stabilized chlorine dioxide. This material is added to the mixture after heat sterilization.

### Example 7

The composition of Example 6, in the form of eye drops, is administered to the eye of a human patient about to undergo a surgical procedure in which the eye is to be exposed to laser energy, in particular, a LASIK surgical procedure.

After the surgical procedure, the patient has reduced pain and/or reduced discomfort and/or reduced eye irritation and/or more rapid recovery from the surgical procedure relative to undergoing an identical surgical procedure including being administered the same composition without the polypeptide analog.

### Example 8

The composition of Example 6, in the form of eye drops, is administered to the eye of a human patient undergoing a surgical procedure in which the eye is to be exposed to laser energy, in particular, a LASIK surgical procedure.

After the surgical procedure, the patient has reduced pain and/or reduced discomfort and/or reduced eye irritation and/or more rapid recovery from the surgical procedure relative to undergoing an identical surgical procedure including being administered the same composition without the polypeptide analog.

### Example 9

The composition of Example 6, in the form of eye drops, is administered to the eye of a human patient substantially immediately after undergoing a surgical procedure in which the eye is to be exposed to laser energy, in particular, a LAS IK surgical procedure.

The patient has reduced pain and/or reduced discomfort and/or reduced eye irritation and/or more rapid recovery from the surgical procedure relative to undergoing an identical surgical procedure including being administered the same composition without the polypeptide analog.

### Example 10

A series of four ophthalmic formulations in accordance with the present invention are prepared by blending the various components (shown in the following table) together.

| | Concentration, % (w/v) | | | |
|---|---|---|---|---|
| Ingredient | A | B | C | D |
| Carboxy | 1.0 | — | — | 0.5 |
| Methylcellulose (CMC) | | | | |
| Glycerol | 0.5 | 0.5 | — | 0.5 |
| Erythritol | 0.25 | 0.25 | 0.75 | 0.75 |
| Boric Acid | 0.60 | 0.60 | 0.60 | 0.60 |
| Sodium Borate | 0.045 | 0.045 | 0.045 | 0.045 |
| Decahydrate | | | | |
| Calcium Chloride | 0.006 | 0.006 | 0.006 | 0.006 |
| Dihydrate | | | | |
| Magnesium Chloride | 0.006 | 0.006 | 0.006 | 0.006 |
| Hexahydrate | | | | |
| Purite7⁽¹⁾ | 0.0075 | 0.0075 | 0.075 | 0.075 |
| Sodium Hydroxide 1N | Adjust pH | Adjust pH | Adjust pH | Adjust pH |
| | to 7.2 | to 7.2 | to 7.2 | to 7.2 |
| Hydrochloric Acid 1N | Adjust pH | Adjust pH | Adjust pH | Adjust pH |
| | to 7.2 | to 7.2 | to 7.2 | to 7.2 |
| Purified water | q.s. ad. | q.s. ad. | q.s. ad. | q.s. ad. |

⁽¹⁾Purite7 is a registered trademark of Allergan, Inc. for stabilized chlorine dioxide. This material is added to the mixture after heat sterilization.

### Example 11

The procedure of Example 10 is repeated to provide the following compositions.

| | Concentration, % (w/v) | | | |
|---|---|---|---|---|
| Ingredient | A | B | C | D |
| Carboxy | 1.0 | — | — | 0.5 |
| Methylcellulose (CMC) | | | | |
| Glycerol | 0.5 | 0.5 | — | 0.5 |
| Xylitol | 0.25 | 0.25 | 0.75 | 0.75 |
| Boric Acid | 0.60 | 0.60 | 0.60 | 0.60 |
| Sodium Borate | 0.045 | 0.045 | 0.045 | 0.045 |
| Decahydrate | | | | |
| Calcium Chloride | 0.006 | 0.006 | 0.006 | 0.006 |
| Dihydrate | | | | |
| Magnesium Chloride | 0.006 | 0.006 | 0.006 | 0.006 |
| Hexahydrate | | | | |
| Purite7⁽¹⁾ | 0.0075 | 0.0075 | 0.075 | 0.075 |
| Sodium Hydroxide 1N | Adjust pH | Adjust pH | Adjust pH | Adjust pH |
| | to 7.2 | to 7.2 | to 7.2 | to 7.2 |
| Hydrochloric Acid 1N | Adjust pH | Adjust pH | Adjust pH | Adjust pH |
| | to 7.2 | to 7.2 | to 7.2 | to 7.2 |
| Purified water | q.s. ad. | q.s. ad. | q.s. ad. | q.s. ad. |

⁽¹⁾Purite7 is a registered trademark of Allergan, Inc. for stabilized chlorine dioxide. This material is added to the mixture after heat sterilization.

### Example 12

The procedure of Example 10 is repeated to provide the following compositions.

| | Concentration, % (w/v) | | | |
|---|---|---|---|---|
| Ingredient | A | B | C | D |
| Carboxy | 1.0 | — | — | 0.5 |
| Methylcellulose (CMC) | | | | |
| Glycerol | 0.5 | 0.5 | — | 0.5 |
| Myo-inositol | 0.25 | 0.25 | 0.75 | 0.75 |
| Boric Acid | 0.60 | 0.60 | 0.60 | 0.60 |
| Sodium Borate | 0.045 | 0.045 | 0.045 | 0.045 |
| Decahydrate | | | | |
| Calcium Chloride | 0.006 | 0.006 | 0.006 | 0.006 |
| Dihydrate | | | | |
| Magnesium Chloride | 0.006 | 0.006 | 0.006 | 0.006 |
| Hexahydrate | | | | |
| Purite7⁽¹⁾ | 0.0075 | 0.0075 | 0.075 | 0.075 |
| Sodium Hydroxide 1N | Adjust pH | Adjust pH | Adjust pH | Adjust pH |
| | to 7.2 | to 7.2 | to 7.2 | to 7.2 |
| Hydrochloric Acid 1N | Adjust pH | Adjust pH | Adjust pH | Adjust pH |
| | to 7.2 | to 7.2 | to 7.2 | to 7.2 |
| Purified water | q.s. ad. | q.s. ad. | q.s. ad. | q.s. ad. |

⁽¹⁾Purite7 is a registered trademark of Allergan, Inc. for stabilized chlorine dioxide. This material is added to the mixture after heat sterilization.

### Example 13

The procedure of Example 10 is repeated to provide the following compositions.

| | Concentration, % (w/v) | | | |
|---|---|---|---|---|
| Ingredient | A | B | C | D |
| Carboxy | 1.0 | — | — | 0.5 |
| Methylcellulose (CMC) | | | | |
| Glycerol | 0.5 | 0.5 | — | 0.5 |
| Carnitine | 0.25 | 0.25 | 0.75 | 0.75 |
| Boric Acid | 0.60 | 0.60 | 0.60 | 0.60 |
| Sodium Borate | 0.045 | 0.045 | 0.045 | 0.045 |
| Decahydrate | | | | |
| Calcium Chloride | 0.006 | 0.006 | 0.006 | 0.006 |
| Dihydrate | | | | |
| Magnesium Chloride | 0.006 | 0.006 | 0.006 | 0.006 |
| Hexahydrate | | | | |
| Purite7⁽¹⁾ | 0.0075 | 0.0075 | 0.075 | 0.075 |
| Sodium Hydroxide 1N | Adjust pH | Adjust pH | Adjust pH | Adjust pH |
| | to 7.2 | to 7.2 | to 7.2 | to 7.2 |
| Hydrochloric Acid 1N | Adjust pH | Adjust pH | Adjust pH | Adjust pH |
| | to 7.2 | to 7.2 | to 7.2 | to 7.2 |
| Purified water | q.s. ad. | q.s. ad. | q.s. ad. | q.s. ad. |

⁽¹⁾Purite7 is a registered trademark of Allergan, Inc. for stabilized chlorine dioxide. This material is added to the mixture after heat sterilization.

### Example 14

The procedure of Example 10 is repeated to provide the following compositions.

| | Concentration, % (w/v) | | | |
|---|---|---|---|---|
| Ingredient | A | B | C | D |
| Carboxy | 1.0 | — | — | 0.5 |
| Methylcellulose (CMC) | | | | |
| Glycerol | 0.5 | 0.5 | — | 0.5 |
| Taurine | 0.25 | 0.25 | 0.75 | 0.75 |
| Boric Acid | 0.60 | 0.60 | 0.60 | 0.60 |
| Sodium Borate | 0.045 | 0.045 | 0.045 | 0.045 |
| Decahydrate | | | | |
| Calcium Chloride | 0.006 | 0.006 | 0.006 | 0.006 |
| Dihydrate | | | | |
| Magnesium Chloride | 0.006 | 0.006 | 0.006 | 0.006 |
| Hexahydrate | | | | |
| Purite7⁽¹⁾ | 0.0075 | 0.0075 | 0.075 | 0.075 |
| Sodium Hydroxide 1N | Adjust pH | Adjust pH | Adjust pH | Adjust pH |
| | to 7.2 | to 7.2 | to 7.2 | to 7.2 |
| Hydrochloric Acid 1N | Adjust pH | Adjust pH | Adjust pH | Adjust pH |
| | to 7.2 | to 7.2 | to 7.2 | to 7.2 |
| Purified water | q.s. ad. | q.s. ad. | q.s. ad. | q.s. ad. |

⁽¹⁾Purite7 is a registered trademark of Allergan, Inc. for stabilized chlorine dioxide. This material is added to the mixture after heat sterilization.

### Example 15

The procedure of Example 10 is repeated to provide the following compositions.

| | Concentration, % (w/v) | | | |
|---|---|---|---|---|
| Ingredient | A | B | C | D |
| Carboxy | 1.0 | — | — | 0.5 |
| Methylcellulose (CMC) | | | | |
| Glycerol | 0.5 | 0.5 | — | 0.5 |
| Betaine⁽²⁾ | 0.25 | 0.25 | 0.75 | 0.75 |
| Boric Acid | 0.60 | 0.60 | 0.60 | 0.60 |
| Sodium Borate | 0.045 | 0.045 | 0.045 | 0.045 |
| Decahydrate | | | | |
| Calcium Chloride | 0.006 | 0.006 | 0.006 | 0.006 |
| Dihydrate | | | | |
| Magnesium Chloride | 0.006 | 0.006 | 0.006 | 0.006 |
| Hexahydrate | | | | |
| Purite7⁽¹⁾ | 0.0075 | 0.0075 | 0.075 | 0.075 |
| Sodium Hydroxide 1N | Adjust pH | Adjust pH | Adjust pH | Adjust pH |
| | to 7.2 | to 7.2 | to 7.2 | to 7.2 |
| Hydrochloric Acid 1N | Adjust pH | Adjust pH | Adjust pH | Adjust pH |
| | to 7.2 | to 7.2 | to 7.2 | to 7.2 |
| Purified water | q.s. ad. | q.s. ad. | q.s. ad. | q.s. ad. |

⁽¹⁾Purite7 is a registered trademark of Allergan, Inc. for stabilized chlorine dioxide. This material is added to the mixture after heat sterilization.
⁽²⁾Betaine is found to be incompatible with the Purite7 preservative. Therefore, no preservative is used. These compositions are useful in single or unit dose applications.

### Example 16

The procedure of Example 10 is repeated to provide the following compositions.

| | Concentration, % (w/v) | | | |
|---|---|---|---|---|
| Ingredient | A | B | C | D |
| Carboxy | 0.5 | — | 0.5⁽³⁾ | — |
| Methylcellulose (CMC) | | | | |
| Glycerol | 0.9 | 0.9 | 0.9 | 0.9 |
| Erythritol | 0.5 | 0.5 | 0.25 | 0.25 |
| Carnitine HCL | 0.1 | 0.25 | 0.1 | 0.25 |
| Boric Acid | 0.45 | 0.45 | 0.45 | 0.45 |
| Sodium Borate | 0.46 | 0.46 | 0.46 | 0.46 |
| Sodium Citrate | 0.1 | 0.1 | 0.1 | 0.1 |
| Potassium Chloride | 0.14 | 0.14 | 0.14 | 0.14 |
| Calcium Chloride | 0.006 | 0.006 | 0.006 | 0.006 |
| Magnesium Chloride | 0.006 | 0.006 | 0.006 | 0.006 |
| Purite7⁽¹⁾ | 0.01 | 0.01 | 0.01 | 0.01 |
| Sodium Hydroxide 1N | Adjust pH | Adjust pH | Adjust pH | Adjust pH |
| | to 7.2 | to 7.2 | to 7.2 | to 7.2 |
| Hydrochloric Acid 1N | Adjust pH | Adjust pH | Adjust pH | Adjust pH |
| | to 7.2 | to 7.2 | to 7.2 | to 7.2 |
| Purified water | q.s. ad. | q.s. ad. | q.s. ad. | q.s. ad. |

⁽¹⁾Purite is a registered trademark of Allergan, Inc. for stabilized chlorine dioxide. This material is added to the mixture after heat sterilization.
⁽³⁾A mixture of 10% by weight high molecular weight carboxylmethyl cellulose having a weight average molecular weight of about 700,000, and 90% by weight medium molecular weight carboxymethyl cellulose having a weight average molecular weight of about 250,000.

### Example 17

Each of the compositions produced in Examples 10 through 16, in the form of eye drops, is administered once a day or more often to the eyes of a patient suffering from dry eye syndrome. Administration may be either in response to or in anticipation of exposure to adverse environmental conditions for example dry or windy environments, low humidity, extensive computer use, and the like. Such administration is substantially similar to that used with conventional artificial tear compositions.

All of the patients, after one week of such administration, are found to have received substantial relief, for example, in terms of reduced pain and/or reduced is irritation and/or enhanced vision and/or enhanced eye appearance, from the effects or symptoms of dry eye syndrome. In addition, those patients who are administered compositions including carboxymethyl cellulose (CMC) are found to have benefited from the anionic character of the CMC and the relatively increased viscosities of such compositions. Such benefits include, without limitation, reduced irritation for longer periods of time after administration, and/or enhanced eye lubrication and/or enhanced protection against adverse effects of cationic species on the ocular surfaces of the patient's eyes.

### Example 18

Each of the compositions produced in Examples 10 through 16 including carboxymethyl cellulose (CMC), in the form of eye drops, is administered to an eye of a different human patient about to undergo a LAS IK surgical procedure.

After the surgical procedure, each of the patients has reduced pain and/or reduced discomfort and/or reduced eye irritation and/or more rapid recovery from the surgical procedure relative to undergoing an identical surgical procedure including being administered the same composition without the carboxymethyl cellulose.

### Example 19

Each if the compositions produced in Examples 10 through 16 including carboxymethyl cellulose, in the form of eye drops, is administered to the eye of a different human patient undergoing a LASIK surgical procedure.

After the surgical procedure, each of the patients has reduced pain and/or reduced discomfort and/or reduced eye irritation and/or more rapid recovery from the surgical procedure relative to undergoing an identical surgical procedure including being administered the same composition without the carboxymethyl cellulose.

### Example 20

Each of the compositions produced in Examples 10 through 16 including carboxymethyl cellulose, in the form of eye drops, is administered to the eye of a different human patient substantially immediately after undergoing a LASIK surgical procedure.

Each patient has reduced pain and/or reduced discomfort and/or reduced eye irritation and/or more rapid recovery from the surgical procedure relative to undergoing an identical surgical procedure including being administered the same composition without the carboxymethyl cellulose.

### Example 21

The following formulations are prepared for use in the following clinical studies.

Concentration,
% (w/v)

| Ingredient | A | B |
|---|---|---|
| Carboxy | 0.5 | 0.5 |
| Methylcellulose (CMC) | | |
| Glycerol | 0.9 | 0.9 |
| Erythritol | 0.25 | 0.25 |
| Carnitine HCL | 0.25 | 0.25 |
| Boric acid | 0.7 | 0.7 |
| Sodium borate | 0.2 | 0.2 |
| decahydrate | | |
| Sodium Citrate | 0.1 | 0.1 |
| Potassium Chloride | 0.14 | 0.14 |
| Calcium Chloride | 0.006 | 0.006 |
| dihydrate | | |
| Magnesium Chloride | 0.006 | 0.006 |
| Purite | 0.01 | — |
| Sodium Hydroxide 1N | Adjust pH | Adjust pH |
| | to 7.2 | to 7.2 |
| Hydrochloric Acid 1N | Adjust pH | Adjust pH |
| | to 7.2 | to 7.2 |
| Purified water | q.s. ad. | q.s. ad. |

The CMC is provided as a 0.325/0.175 mixture of medium/high molecular weight polymers

A secondary analysis was done on data collected from 2 multi center, randomized, controlled clinical trials in which subjects with dry eye signs and symptoms, used Optive™ Lubricant Eye props (Example 21(a)) for 90 days (Trial 1) and Optive™ Sensitive Preservative Free Lubricant Eye props (Example 21(b)) for 30 days (Trial 2). Each subject was dosed with 1 to 2 drops per eye, as needed, but at least twice daily.

The key inclusion criteria: male or female adults (18 years of age) with dry eye symptoms, as evidenced by either a reduced Schirmer or Tear Break-up score and currently used artificial tears.

The key exclusion criteria was whether the subject currently used other topical ophthalmic medications.

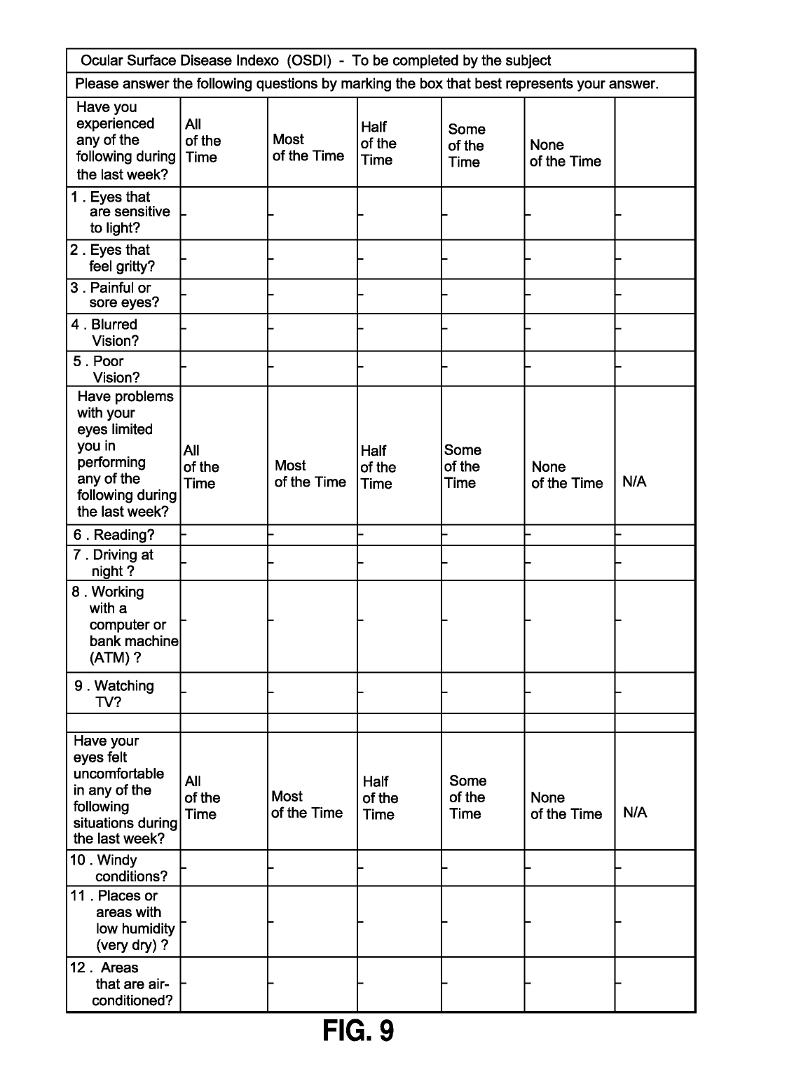

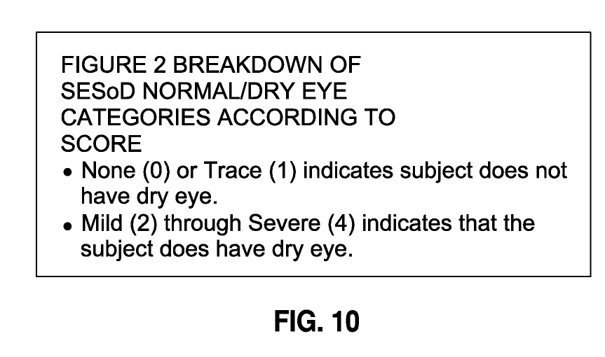

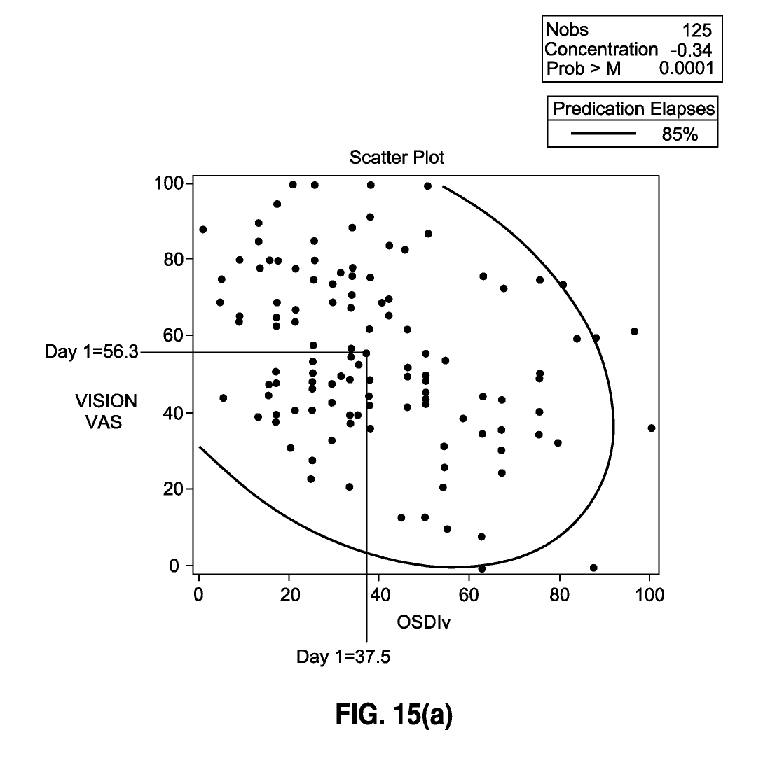

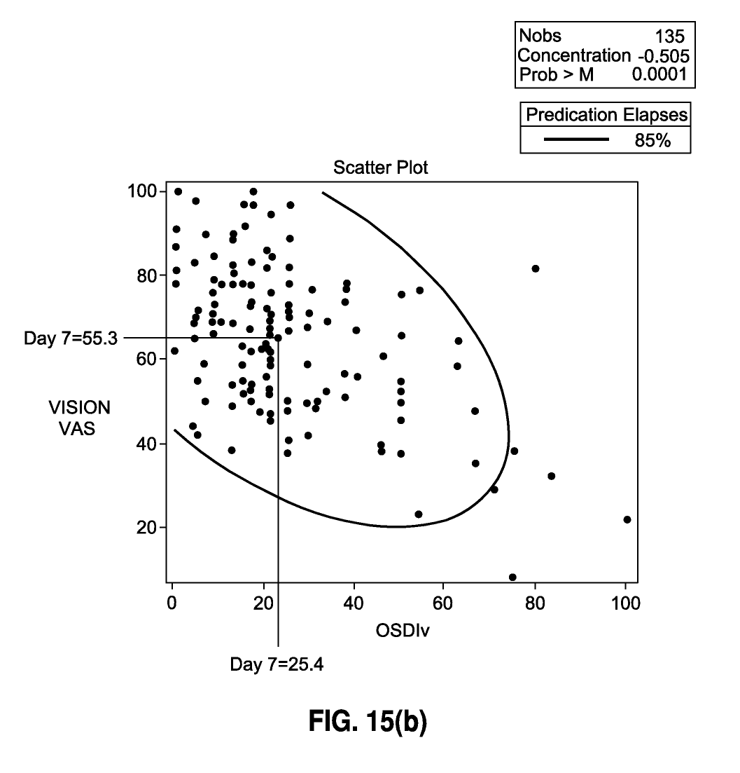

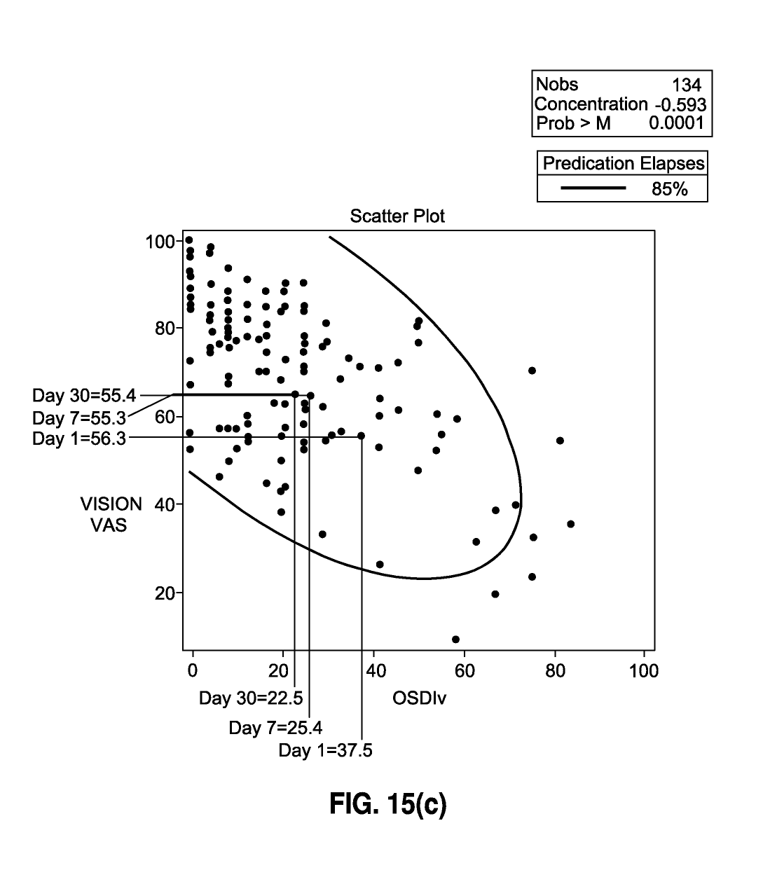

The subjects were directly assigned to a study treatment from their prior product. Testing of within-group change from baseline was performed using a paired t-test and is shown in FIGS. 9, 10, 14 and 15: FIGS. 15a, b and c show the results of testing of correlation based on a t-approximation.

Subjective Variables of Symptoms and Visual Function:

Overall Dry Eye Symptoms were measured as follows: Ocular Surface Disease Index (OSDI), Subjective Evaluation of Symptoms of Dryness (SESoD) and Dryness Comfort Level Visual Analog Scale (Dryness VAS)

Visual Symptoms were measured as follows: OSDI Subscale of Vision Related Function Questions (OSDI_{V}), Current Visual Quality VAS (Vision VAS)

FIG. 9 reproduces the OCULAR SURFACE DISEASE INDEX© (OSDI) questionnaire of ALLERGAN, INC. that was used in these clinical trials.

The OSDI is a validated 12-item patient-reported outcomes questionnaire designed to provide an assessment of various symptoms, related visual functions and environmental triggers of dry eye.

The blue outline above shows that the OSDI contains a subscale of vision-related function questions (OSDIᵥ).

Questions are scored on a 0 to 4 Likert-type scale (0=None of the time, 1=Some of the time, 2=Half of the time, 3=Most of the time, 4=All of the time).

Overall and subscale OSDI scores are calculated using the same formula and range from 0 (no disability) to 100 (complete disability).

FIG. 10 shows the Breakdown of SESoD normal/Dry Eye categories according to score.

None (0) or Trace (1) indicates subject does not have dry eye.

Mild (2) through Severe (4) indicates that the subject does have dry eye.

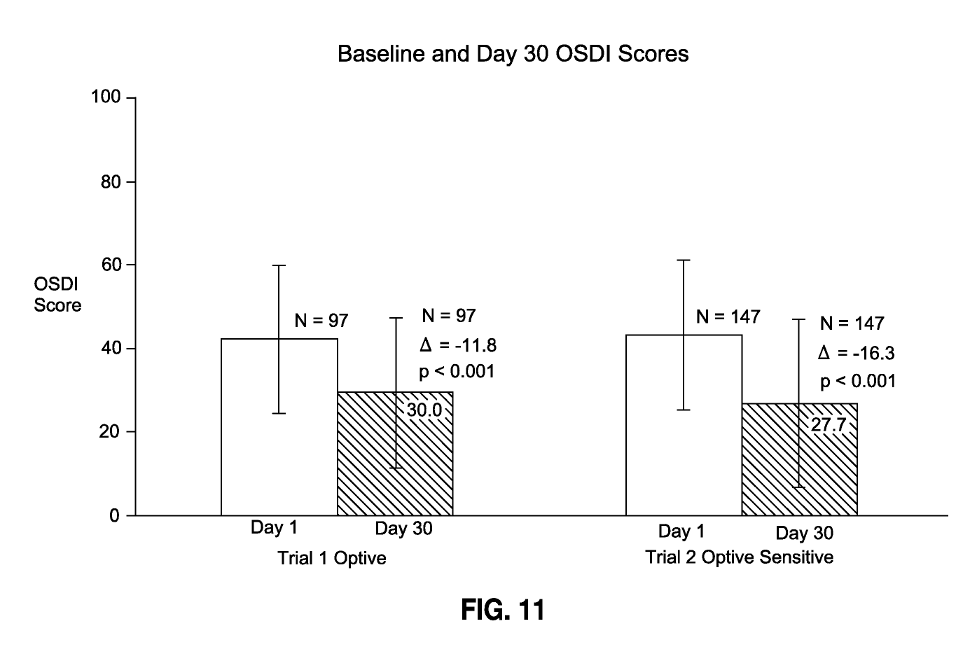

The results for the baseline and day 30 OCULAR SURFACE DISEASE INDEX (OSDI) scores are reported in FIG. 11

In both studies, there was a clinically and statistically significant improvement from baseline (Day 1) in the mean OSDI score at Day 30.

This indicates that various ocular symptoms and related visual functions improved after 30 days with the use of the compositions of Example 21. Note: Last observation carried forward (LOCF) was used to impute for missing values at Day 30.

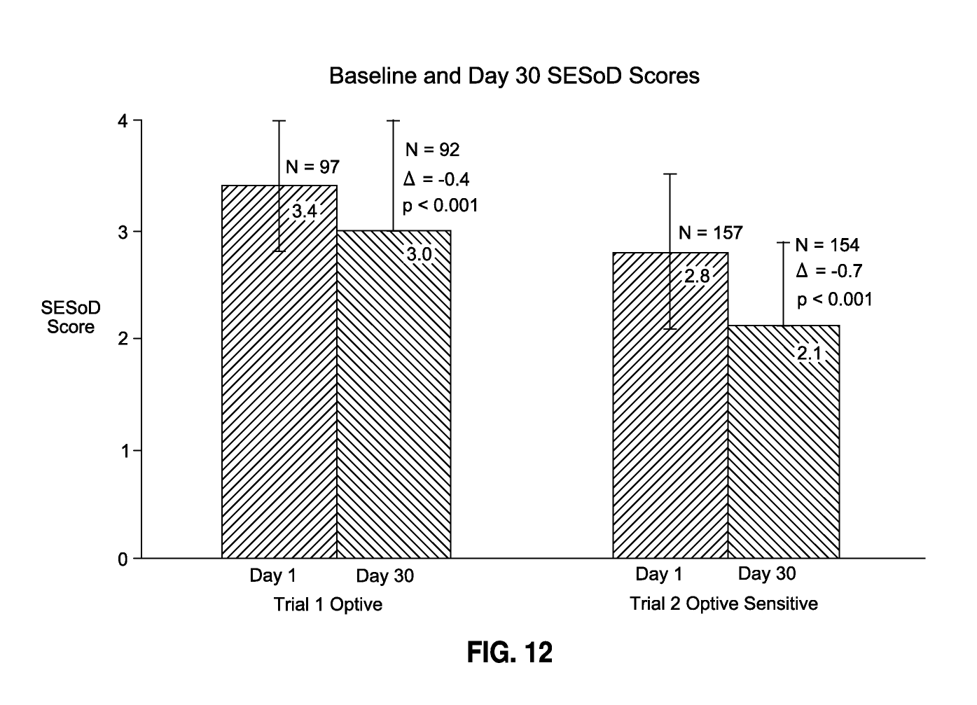

The results of baseline and day 30 SUBJECTIVE EVALUATION OF SYMPTOM OF DRYNESS (SESoD) scores are reported in FIG. 12.

In both clinical studies, there was a statistically significant improvement from baseline (Day 1) in the mean SESoD score, at Day 30.

The mean change in SESoD scores is consistent in direction with the mean change from baseline reported for OSDI.

Subjective Evaluation of Symptom of Dryness, is a 5-point 0 to 4 single variable for subjective grading of severity of dry eye symptoms (4 is worse symptoms).⁶

The SESoD can be used to quickly differentiate “normal” from dry (FIG. 10), classify and track treatment response and has also been recently extensively tested as a screening tool for dry eye clinical trials.

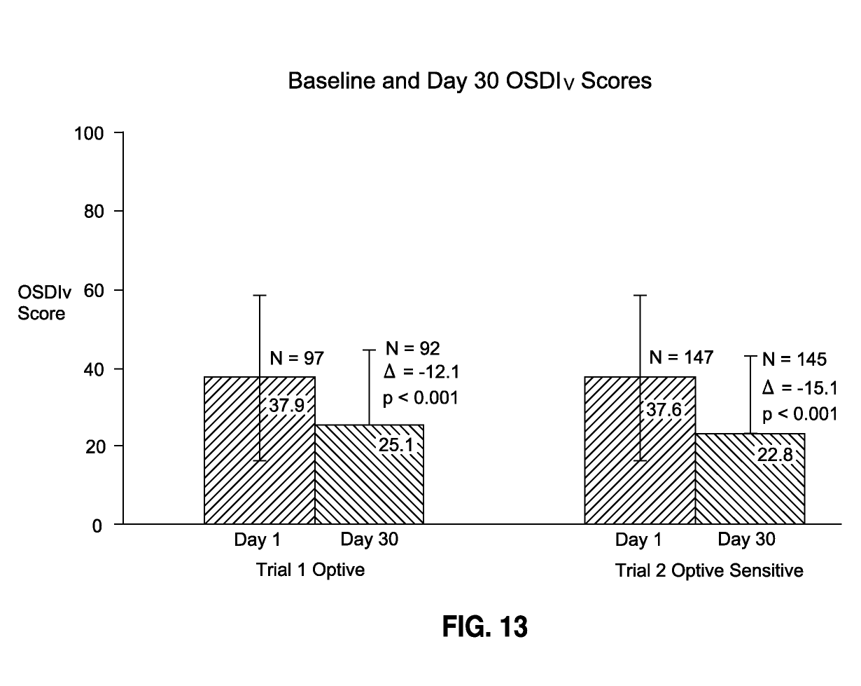

The results of baseline AND Day 30 SUBJECTIVE EVALUATION of OSDI_{V} are reported in FIG. 13.

In both clinical studies, there was a clinically and statistically significant improvement from baseline (Day 1) in the mean OSDI_{V} vision-related function subscale score, at Day 30.

This indicates that visual symptoms improved within 30 days with use of the composition of Example 21(a).

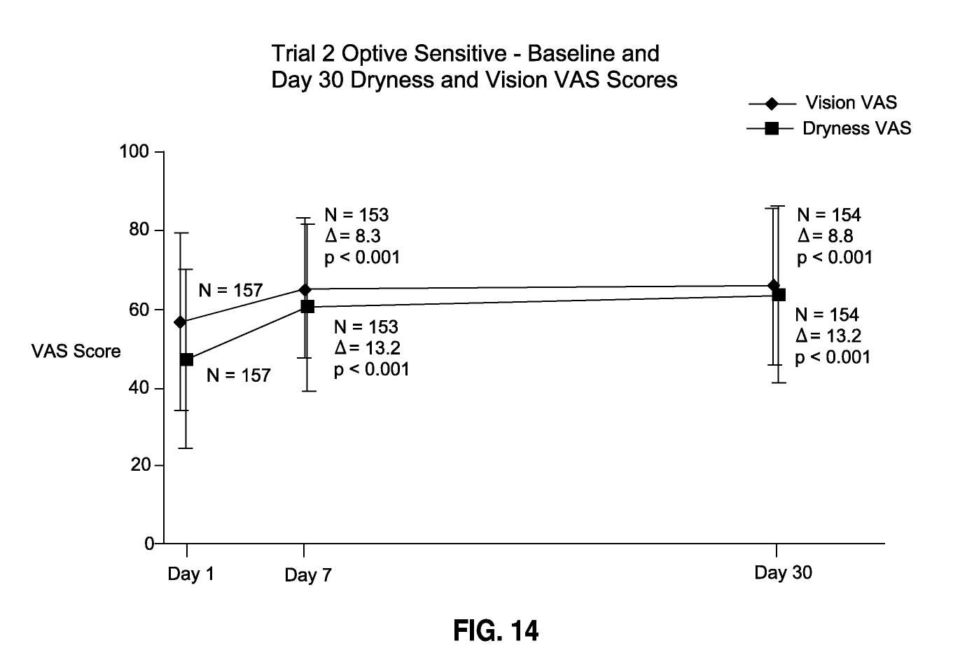

The scores for dryness and vision (VAS) for the second clinical trial of OPTIVE SENSITIVE at baseline and Day 30 are reported in FIG. 14. An improvement in overall vision quality was observed, consistent with improved OSDI_{V}, supporting reduced visual symptoms with use of the composition of Example 21(b).

Consistent with the “Baseline and Day 30 OSDI Scores” graph, Dryness VAS scores demonstrated that there was a clinically significant improvement in the subjective evaluation of dryness severity after 30 days with the use of the compositions of Example 21.

Dryness severity and vision quality were measured using the Current Comfort Level Assessment—a four item subjective questionnaire that captures subject's “real time” overall and ocular comfort at the time of each visit.

Subject responses were captured on anchored visual analog scales (VAS).

Dryness Severity VAS:

Question: In thinking of your eyes at this moment, do you have any dryness, discomfort or irritation?

is Anchors: 0=Yes, could not be worse; 100=No, none at all.

Vision Quality VAS:

Question: How would you rate the overall quality of your vision during the past 2-3 hours prior to your visit today?

Anchors: 0=Very poor, has never been worse; 100=Excellent, has never been better.

FIGS. 15a, b and c show the correlation between OSDI_{V} and vision (VAS) from Clinical Trial 2 of OPTIVE SENSITIVE (Example 21(b).)

A moderate to strong relationship exists between OSDI_{V} and the Vision VAS score at Days 1, 7 and 30.

Use of the compositions of Example 21 for 7 and 30 days shows improvement in both variables with the relationship between OSDI_{V} and VAS intact.

A broad subjective improvement occurred as demonstrated by the shift in the center of the ellipse from Day 1 to Day 30.

As subjects show improvement ceiling effects occur in OSDI_{V}.

The following conclusions may be drawn from the above.

Subjects with dry eye complaints typically have visual problems.

Both dry eye discomfort and visual symptoms improved within 30 days with use of the compositions used in the method of the present invention, e.g. the compositions of Example 21.

The correlation between OSDI_{V} and Vision VAS scores support the usefulness of VAS in evaluating visual symptoms.

Consistent usage of the compositions utilized in the method of this invention rapidly improves the ocular surface, thereby increasing subject's comfort level and improving his visual symptoms.

In particular, OSDI scores improved from 42.4±17.8 to 30.0±18.2 and from 43.0±18.5 to 27.7±20.1 SESoD improved from 3.4±0.6 to 3.0±1.0 and from 2.8±0.7 to 2.1±0.8. Dryness VAS improved from 48.3±21.8 to 61.6±25.1 and from 47.4±22.8 to 63.3±22.7. Visual symptoms also improved within 1 week in both trials. At Day 30, OSDIᵥ scores improved from 37.9±21.3 to 25.1±19.4 and from 37.6±21.5 to 22.8±20.4. Vision VAS collected in trial 2 improved from 56.8±22.4 to 65.4±20.1. All changes were significant (p<0.001). The Spearman Correlation Coefficient between OSDIᵥ and Vision VAS was r=−0.433 at Day 7 (p<0.001) and r=−0.528 at Day 30 (p<0.001).

Thus, both dry eye discomfort and visual symptoms improved within 30 days with use of the composition of Example 21.

While this invention has been described with respect to various specific examples and embodiments, it is to be understood that the invention is not limited thereto and that it can be variously practiced within the scope of the following claims.

What is claimed is:

1. A method of improving visual acuity of a person in need of such improvement, the method comprising administering to the person an effective amount of a composition comprising erythritol, glycerol, carnitine, and an aqueous carrier.

2. The method of claim 1 wherein the person suffers from Dry Eye Syndrome.

3. The method of claim 1 wherein the material is effective, when the composition is administered to an eye, to allow an ocular surface of the eye to better tolerate a hypertonic condition on the ocular surface relative to an identical method without the material.

4. The method of claim 1 wherein said composition has an osmolality in a range of about 300 to about 1000 mOsmols/kg.

5. The method of claim 1 wherein the composition has an osmolality in a range of about 300 to about 600 mOsmols/kg.

6. The method of claim 1 wherein said composition is substantially free of is inorganic osmolytes.

7. The method of claim 1 wherein the total amount of erythritol, glycerol, and carnitine is in a range of about 0.01% (w/v) to about 3% (w/v).

8. A method of treating an eye of a human or animal comprising administering a composition of claim 1 to an eye of a human or animal, thereby providing at least one additional benefit to the eye besides improving visual acuity.

9. The method of claim 8 wherein said additional benefit is relieving the discomfort of Dry Eye.

10. A method of improving the visual acuity of a person in need of such improvement, the method comprising topically administering to the person, in an effective amount, an ophthalmic composition comprising: an aqueous carrier component; a tonicity component in an amount effective to provide the method with a desired osmolality, the tonicity component comprising a combination of compatible solute agents, wherein the combination of compatible solute agents comprises two polyol components and one amino acid component and wherein the polyol components are erythritol and glycerol and the amino acid component is carnitine; and a polyanionic component in an amount effective, when the composition is administered to a human or animal eye, to reduce at least one adverse effect of a polycationic material on an ocular surface of a human or animal eye relative to an identical composition without the polyanionic component.

11. The method of claim 10 wherein the tonicity component is effective, when the composition is administered to an eye, to allow an ocular surface of the eye to better tolerate a hypertonic condition on the ocular surface relative to an identical composition without the compatible solute component.

12. The method of claim 10 wherein the composition has an osmolality in a range of about 300 to about 1000 mOsmols/kg.

13. The method of claim 10 wherein the composition has an osmolality in a range is of about 300 to about 600 mOsmols/kg.

14. The method of claim 10 wherein the polyanionic component is a polymeric polyanionic component.

15. The method of claim 10 wherein the polyanionic component is present in an amount in a range of about 0.1% (w/v) to about 10% (w/v) of the method.

16. The method of claim 10 wherein the polyanionic component is selected from the group consisting of anionic cellulose derivatives, hyaluronic acid, anionic starch derivatives, poly methacrylic acid, poly methacrylic acid derivatives, polyphospazene derivatives, poly aspartic acid, poly aspartic acid derivatives, gelatin, alginic acid, alginic acid derivatives, poly acrylic acid, poly acrylic acid derivatives and mixtures thereof.

17. The method of claim 10 wherein the polyanionic component is carboxymethyl cellulose.

18. The method of claim 10 wherein the polyanionic component is selected from the group consisting of polyanionic peptides, polyanionic peptide analogs, portions of polyanionic peptide analogs, carboxymethyl-substituted polymers of sugars and mixtures thereof.

19. The method of claim 10 wherein the polyanionic component comprises an agent having an activity which mimics an activity of a pro-piece of Major Basic Protein.

20. The method of claim 10 wherein the polyanionic component comprises an agent selected from the group consisting of polypeptide analogs of a Major Basic Protein pro-piece sequence, polypeptide analogs of a portion of a Major Basic Protein pro-piece sequence and mixtures thereof.

21. The method of claim 20 wherein the person suffers from Dry Eye Syndrome.

22. A method of treating an eye of a human or animal comprising administering the composition of claim 10 to an eye of a human or animal, thereby providing at least one additional benefit to the eye besides improving visual acuity.

23. The method of claim 22 wherein the additional benefit is relieving the discomfort of Dry eye.

24. The method of claim 10 wherein the ophthalmic composition has the following composition:
Concentration,
% (w/v)

| Ingredient | A | B |
|---|---|---|
| Carboxy | 0.5 | 0.5 |
| Methylcellulose (CMC) | | |
| Glycerol | 0.9 | 0.9 |
| Erythritol | 0.25 | 0.25 |
| Carnitine HCL | 0.25 | 0.25 |
| Boric acid | 0.7 | 0.7 |
| Sodium borate | 0.2 | 0.2 |
| decahydrate | | |
| Sodium Citrate | 0.1 | 0.1 |
| Potassium Chloride | 0.14 | 0.14 |
| Calcium Chloride | 0.006 | 0.006 |
| dihydrate | | |
| Magnesium Chloride | 0.006 | 0.006 |
| Purite | 0.01 | — |
| Sodium Hydroxide 1N | Adjust pH | Adjust pH |
| | to 7.2 | to 7.2 |
| Hydrochloric Acid 1N | Adjust pH | Adjust pH |
| | to 7.2 | to 7.2 |
| Purified water | q.s. ad. | q.s. ad. |
